(19) United States
(12) Patent Application Publication
(10) Pub. No.: US 2025/0195594 A1
(43) Pub. Date: Jun. 19, 2025

(54) Ingestible Nutritive Compositions With Prebiotics, Probiotics, and Postbiotics

(71) Applicant: First Day Life Inc., Austin, TX (US)

(72) Inventors: Bridgette Park, Granada Hills, CA (US); Shuyan Li, Austin, TX (US)

(21) Appl. No.: 18/544,233

(22) Filed: Dec. 18, 2023

(51) Int. Cl. A61K 36/185; A61K 9/00; A61K 35/00; A61K 35/742; A61K 35/747; A61P 1/00; A61P 1/02; A61P 1/10; A61P 1/12; A61P 1/14; A61P 3/06; A61P 15/02; A61P 17/00; A61P 37/04

(52) U.S. Cl. CPC A61K 36/185; A61K 9/0053; A61K 35/742; A61K 35/747; A61P 1/00; A61P 1/02; A61P 1/10; A61P 1/12; A61P 1/14; A61P 3/06; A61P 15/02; A61P 17/00; A61P 37/04; A61K 2035/115

(57) ABSTRACT

Compositions including prebiotics, probiotics, and postbiotics for oral administration and methods of using such compositions for improving health of subjects are provided.

### BACKGROUND OF THE INVENTION

The present invention generally relates to nutritional compositions comprising prebiotics, probiotics, and postbiotics for oral administration and methods for using the oral compositions in improving health of subjects (e.g., human subjects).

### BRIEF SUMMARY OF THE INVENTION

In an aspect of the invention, an orally ingestible nutritional composition for improving health is provided. The composition comprises an amount of gold kiwifruit powder, an amount of Bacillus coagulans, an amount of Bacillus subtilis, and an amount of heat-treated Lactobacillus plantarum. The gold kiwifruit powder within the composition may include or consist essentially of Livaux®. The Bacillus coagulans within the composition may include or consist essentially of Unique IS-2. The heat-treated Lactobacillus plantarum within the composition may include or consist essentially of L-137. The Bacillus subtilis within the composition may include or consist essentially of BS50.

In some embodiments, components of the inventive composition may be combined at a w/w ratio of between about 5:1 and about 20:1 gold kiwifruit powder:Bacillus coagulans. In some embodiments, components of the inventive composition may be combined at a w/w ratio of between about 5:1 and about 20:1 gold kiwifruit powder:Bacillus subtilis. In some embodiments, components of the inventive composition may be combined at a w/w ratio of between about 3:1 and about 7:1 gold kiwifruit powder:Lactobacillus plantarum.

In some embodiments, components of the inventive composition may be combined at a w/w ratio of between about 1.5:1 and about 3.5: Lactobacillus plantarum:Bacillus coagulans. In some embodiments, components of the inventive composition may be combined at a w/w ratio of between about 1.5:1 and about 3.5:1 Lactobacillus plantarum:Bacillus subtilis.

In some embodiments, components of the inventive composition may be combined at a w/w ratio of between about 0.5:1 and about 1.5:1 Bacillus coagulans:Bacillus subtilis.

In some embodiments, the orally ingestible composition may take the form of gummies, tablets, capsules, liquids, suspensions, chewables, soft gels, sachets, powders, syrups, liquid suspensions, emulsions, or other solutions. In particular embodiments, the orally ingestible composition is in the form of an individual dosage form. The individual dosage form may take the form of a gummy, a tablet, a capsule, or a chew. In particular embodiments, the individual dosage form is a gummy.

In further embodiments, an individual oral dosage form may comprise between about 75 mg and 700 mg gold kiwifruit powder; between about 5 mg and 140 mg heat-treated Lactobacillus plantarum; between about 5 mg and 90 mg Bacillus subtilis; and between about 5 mg and 90 mg Bacillus coagulans. In particular embodiments, the individual oral dosage form comprises about 125 mg gold kiwifruit powder; about 25 mg heat-treated 11 Lactobacillus plantarum; about 10 mg Bacillus subtilis; and about 10 mg Bacillus coagulans. In particular embodiments, an individual oral dosage form may comprise an amount of gold kiwifruit powder, an amount of heat-treated Lactobacillus plantarum, between about 500 million CFUs and 9 billion CFUs Bacillus coagulans, and between about 500 million CFUs and 9 billion CFUs Bacillus subtilis. In particular embodiments, the individual oral dosage form comprises an amount of gold kiwifruit powder, an amount of heat-treated Lactobacillus plantarum, about 1 billion CFUs Bacillus coagulans, and about 1 billion CFUs Bacillus subtilis.

In an aspect, there is a method for administering orally ingestible compositions to a subject in accordance with the disclosure. In some embodiments, the subject is a mammal. In some embodiments, the subject is a human. In some embodiments, the subject is an adult human. In particular embodiments, the subject is an adult human female. In particular embodiments, the subject is an adult human male. In particular embodiments, the subject is an elderly adult human. In still further embodiments, the subject is an elderly adult female. In still further embodiments, the subject is an elderly adult male. In some embodiments, the subject is a human child under the age of 18.

Compositions according to embodiments herein may be administered a single time, daily on a continual basis, or multiple times per week depending on subject needs, subject body size, and on safety. Dosing may occur once, or only a weekly or monthly basis. Dosage level and frequency of administration protocols, compositions, as well as potential use of additional therapeutic or nutritive agents in combination can be adjusted during the course of administration. In an embodiment, compositions in accordance with the present inventions can be administered daily to a subject. In a particular embodiment, compositions in accordance with the present inventions can be administered multiple times per day to a subject.

In some embodiments, the subject receives, via oral administration of compositions in accordance with the disclosure herein, between about 75 mg and 2500 mg of gold kiwifruit powder per day.

In some embodiments, the subject receives, via oral administration of compositions in accordance with the disclosure herein, between about 5 mg and 1000 mg of 8 heat-treated Lactobacillus plantarum per day.

In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day.

In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In an embodiment, a method of increasing the amount of Faecalibacterium prausnitzii within a digestive tract of a subject is provided. The method comprises orally administering, to the subject, an orally ingestible composition in accordance with the disclosure herein. In particular embodiments, the subject is a human. In particular embodiments, the subject is a human adult. In further embodiments, the subject is an adult human female. In some embodiments, the method of increasing the amount of Faecalibacterium prausnitzii within a digestive tract of a subject comprises orally delivering to the subject an effective amount or concentration of one or more of gold kiwifruit powder, Lactobacillus plantarum, Bacillus subtilis, and Bacillus coagulans to achieve an increase in the amount of Faecalibacterium prausnitzii within the digestive tract of the subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In an embodiment, a method of reducing constipation or relieving symptoms of constipation in a subject is provided. The method comprises orally administering, to the subject, an orally ingestible composition in accordance with the disclosure herein. In particular embodiments, the subject is a human. In particular embodiments, the subject is a human adult. In further embodiments, the subject is an adult human female.

In some embodiments, the method of reducing constipation or relieving symptoms of constipation in a subject comprises orally delivering to the subject an effective amount or concentration of one or more of gold kiwifruit powder, Lactobacillus plantarum, Bacillus subtilis, and Bacillus coagulans to achieve a reduction in constipation or relief of symptoms of constipation in the subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day. In some embodiments of the method of reducing constipation or relieving symptoms of constipation in a subject, the subject's frequency of defecation is increased post-administration of the orally ingestible composition. In some embodiments, the subject's frequency of defecation is increased post-administration to at least three bowel movements per week.

In some embodiments of the method of reducing constipation or relieving symptoms of constipation in a subject, the subject's stools are softened and/or improved in consistency post-administration of the orally ingestible composition as measured by the Bristol stool form scale.

In some embodiments of the method of reducing constipation or relieving symptoms of constipation in a subject, the subject's pain during defecation is reduced post-administration of the orally ingestible composition as measured by the constipation scoring system (CSS) scale.

In some embodiments of the method of reducing constipation or relieving symptoms of constipation in a subject, the subject's abdominal pain associated with constipation is reduced post-administration of the orally ingestible composition as measured by the constipation scoring system (CSS) scale.

In an embodiment, a method of improving cholesterol levels in a subject is provided. The method comprises orally administering, to the subject, an orally ingestible composition in accordance with the disclosure herein. In particular embodiments, the subject is a human. In particular embodiments, the human subject has hyperlipidemia (i.e., having serum cholesterol levels of greater than 200 mg/dL). In particular embodiments, the subject is a human adult. In further embodiments, the subject is an adult human female. In some embodiments, the method of improving cholesterol levels in a subject comprises orally delivering to the subject an effective amount or concentration of one or more of gold kiwifruit powder, Lactobacillus plantarum, Bacillus subtilis, and Bacillus coagulans to achieve an improvement in cholesterol levels in the subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day. In some embodiments of the method of improving cholesterol levels in a subject, the subject's serum HDL is increased post-administration of the orally ingestible composition.

In some embodiments of the method of improving cholesterol levels in a subject, the subject's serum LDL is decreased post-administration of the orally ingestible composition.

In some embodiments of the method of improving cholesterol levels in a subject, the subject's ratio of serum HDL to serum LDL is increased post-administration. In some embodiments, the subject's total serum cholesterol is decreased post-administration of the orally ingestible composition.

In an embodiment, a method of reducing bacterial vaginosis symptoms in a human female subject is provided. The method comprises orally administering, to the human female subject, an orally ingestible composition in accordance with the disclosure herein. In particular embodiments, the subject is an adult human female. In some embodiments of the method of reducing bacterial vaginosis symptoms in a human female subject, the method comprises orally delivering to the subject an effective amount or concentration of one or more of gold kiwifruit powder, Lactobacillus plantarum, Bacillus subtilis, and Bacillus coagulans to reduce bacterial vaginosis symptoms in the subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day. In some embodiments, the human female subject is undergoing antibiotic treatment for bacterial vaginosis.

In an embodiment, a method of reducing inflammation caused by plaque-induced gingivitis in a subject is provided. The method comprises orally administering, to the subject, an orally ingestible composition in accordance with the disclosure herein. In particular embodiments, the subject is a human. In particular embodiments, the subject is a human adult. In further embodiments, the subject is an adult human female. In some embodiments, the method of reducing inflammation caused by plaque-induced gingivitis in a subject comprises orally delivering to the subject an effective amount or concentration of one or more of gold kiwifruit powder, Lactobacillus plantarum, Bacillus subtilis, and Bacillus coagulans to achieve reduction in inflammation caused by plaque-induced gingivitis in the subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments of the method of reducing inflammation caused by plaque-induced gingivitis in a subject, the subject's salivary glutathione peroxidase activity is decreased post-administration of the orally ingestible composition.

In an embodiment, a method of reducing incidence of hepatic encephalopathy in a human subject suffering from liver cirrhosis is provided. The method comprises orally administering, to the subject, an orally ingestible composition in accordance with the disclosure herein. In particular embodiments, the subject is a human adult. In further embodiments, the subject is an adult human female. In some embodiments, the method of reducing incidence of hepatic encephalopathy in a human subject suffering from liver cirrhosis comprises orally delivering to the subject an effective amount or concentration of one or more of gold kiwifruit powder, Lactobacillus plantarum, Bacillus subtilis, and Bacillus coagulans to achieve reduction in incidence of hepatic encephalopathy in the human subject suffering from liver cirrhosis. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments of the method of reducing incidence of hepatic encephalopathy in a human subject suffering from liver cirrhosis, the frequency of hospital admissions of the human subject suffering from liver cirrhosis is decreased post-administration of the orally ingestible composition.

In some embodiments of the method of reducing incidence of hepatic encephalopathy in a human subject suffering from liver cirrhosis, the plasma ammonia levels of the human subject suffering from liver cirrhosis are decreased post-administration of the orally ingestible composition.

In an embodiment, a method of improving digestive health in a human subject suffering from acute diarrhea is provided. The method comprises orally administering, to the subject, an orally ingestible composition in accordance with the disclosure herein. In particular embodiments, the subject is a human adult. In further embodiments, the subject is an adult human female. In some embodiments, the method of improving digestive health in a human subject suffering from acute diarrhea comprises orally delivering to the subject an effective amount or concentration of one or more of gold kiwifruit powder, Lactobacillus plantarum, Bacillus subtilis, and Bacillus coagulans to achieve improvement in digestive health in a human subject suffering from acute diarrhea. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments of the method of improving digestive health in a human subject suffering from acute diarrhea, the incidence frequency of diarrhea and/or associated symptoms is reduced in the human subject suffering from acute diarrhea post-administration of the orally ingestible composition.

In some embodiments of the method of improving digestive health in a human subject suffering from acute diarrhea, abdominal pain of the human subject suffering from acute diarrhea is reduced post-administration of the orally ingestible composition.

In some embodiments of the method of improving digestive health in a human subject suffering from acute diarrhea, the incidence frequency of defecation is reduced in the human subject suffering from acute diarrhea post-administration of the orally ingestible composition.

In an embodiment, a method of alleviating irritable bowel syndrome (IBS) symptoms in a human subject suffering from IBS is provided. The method comprises orally administering, to the subject, an orally ingestible composition in accordance with the disclosure herein. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve alleviation of IBS symptoms in the human subject suffering from IBS. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments of the method of alleviating irritable bowel syndrome (IBS) symptoms in a human subject suffering from IBS, the abdominal pain and/or discomfort of the human subject suffering from IBS is reduced post-administration of the orally ingestible composition.

In some embodiments of the method of alleviating irritable bowel syndrome (IBS) symptoms in a human subject suffering from IBS, the incidence frequency of complete spontaneous bowel movements is increased post-administration in the human subject suffering from IBS.

In some embodiments of the method of alleviating irritable bowel syndrome (IBS) symptoms in a human subject suffering from IBS, the abdominal bloating of the human subject suffering from IBS is reduced post-administration of the orally ingestible composition.

In an embodiment, a method of reducing burping in a human subject is provided. The method comprises orally administering, to the subject, an orally ingestible composition in accordance with the disclosure herein. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve reduction in burping of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In an embodiment, a method of reducing flatulence in a human subject is provided. The method comprises orally administering, to the subject, an orally ingestible composition in accordance with the disclosure herein. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve reduction in flatulence of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In an embodiment, a method of supporting protein digestion in a human subject is provided. The method comprises orally administering, to the subject, an orally ingestible composition in accordance with the disclosure herein. In particular embodiments, the subject is a human adult. In further embodiments, the subject is an adult human female. In further embodiments, the subject is an adult human male. In some embodiments, the method comprises orally delivering to the subject an effective amount or concentration of one or more of gold kiwifruit powder, Lactobacillus plantarum, Bacillus subtilis, and Bacillus coagulans to support protein digestion in the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In an embodiment, a method of improving the immune response of a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to improve the immune response of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments of the method of improving the immune response of a human subject, the serum IL-12 is increased in the human subject post-administration of the orally ingestible composition.

In some embodiments of the method of improving the immune response of a human subject, the serum IFN-β is increased in the human subject post-administration of the orally ingestible composition.

In some embodiments of the method of improving the immune response of a human subject, T-cell activity is increased in the human subject post-administration of the orally ingestible composition.

In some embodiments of the method of improving the immune response of a human subject, incidence frequency of upper respiratory tract infections is decreased in the human subject post-administration of the orally ingestible composition.

In some embodiments of the method of improving the immune response of a human subject, hepatic inflammation is decreased in the human subject post-administration of the orally ingestible composition.

In some embodiments of the method of improving the immune response of a human subject, serum aspartate transaminase is decreased in the human subject post-administration of the orally ingestible composition.

In some embodiments of the method of improving the immune response of a human subject, serum alanine transaminase is decreased in the human subject post-administration of the orally ingestible composition.

In some embodiments of the method of improving the immune response of a human subject, seasonal increase in total cholesterol in the human subject is suppressed post-administration of the orally ingestible composition.

In some embodiments of the method of improving the immune response of a human subject, seasonal increase in serum LDL in the human subject is suppressed post-administration of the orally ingestible composition.

In some embodiments of the method of improving the immune response of a human subject, activation of immune cells in the digestive tract of the human subject is increased post-administration of the orally ingestible composition.

In some embodiments of the method of improving the immune response of a human subject, the Th1:Th2 ratio is increased in the human subject post-administration of the orally ingestible composition.

In some embodiments of the method of improving the immune response of a human subject, the incidence frequency of colds and cold-like illness in the human subject is decreased post-administration of the orally ingestible composition.

In some embodiments of the method of improving the immune response of a human subject, the incidence frequency of influenza and influenza-like illness in the human subject is decreased post-administration of the orally ingestible composition.

In an embodiment, a method of improving skin health in a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In particular embodiments, the human subject is suffering from dry skin. In some embodiments, the method comprises orally delivering to the subject an effective amount or concentration of one or more of gold kiwifruit powder, Lactobacillus plantarum, Bacillus subtilis, and Bacillus coagulans to improve the skin health of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments of the method of improving skin health in a human subject, water content within skin of the human subject experiences a greater increase compared to placebo post-administration of the orally ingestible composition. In some embodiments, the skin of the human subject is non-facial body skin. In particular embodiments, the body skin is forearm skin.

In some embodiments of the method of improving skin health in a human subject, transepidermal water loss from skin of the human subject has a greater reduction compared to placebo post-administration of the orally ingestible composition. In some embodiments, the skin of the human subject is facial skin.

In an embodiment, a method of improving chronic periodontitis symptoms in a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In particular embodiments, the human subject is suffering from chronic periodontitis. In some embodiments, the method comprises orally delivering to the subject an effective amount or concentration of one or more of gold kiwifruit powder, Lactobacillus plantarum, Bacillus subtilis, and Bacillus coagulans to improve the chronic periodontitis symptoms of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In an embodiment, a method of improving protein and amino acid absorption in a human subject is provided. The method comprises orally administering, to the subject, an orally ingestible composition in accordance with the disclosure herein. In particular embodiments, the subject is a child under the age of 18. In particular embodiments, the subject is a human adult. In further embodiments, the subject is an adult human female. In further embodiments, the human subject is an adult human male. In some embodiments, the method comprises orally delivering to the subject an effective amount or concentration of one or more of gold kiwifruit powder, Lactobacillus plantarum, Bacillus subtilis, and Bacillus coagulans to improve the protein and amino acid absorption of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments of the method of improving protein and amino acid absorption in a human subject, plasma amino acids in the human subject are increased post-administration of the orally ingestible composition in accordance with the disclosure herein. In particular embodiments, the increased plasma amino acids comprise branched-chain amino acids.

In some embodiments, the method of improving protein and amino acid absorption further comprises orally administering a protein-containing food to the human subject. In some embodiments, the protein-containing food comprises whey protein. In particular embodiments, muscle strength of the human subject has a greater increase post-administration of the orally ingestible composition and the protein-containing food in comparison to muscle strength of human subjects consuming only the protein-containing food.

In an embodiment, a method of improving oral health and reducing tooth decay in a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In further embodiments, the human subject is an adult human male. In some embodiments, the method comprises orally delivering to the subject an effective amount or concentration of one or more of gold kiwifruit powder, Lactobacillus plantarum, Bacillus subtilis, and Bacillus coagulans to improve the oral health of and reduce tooth decay in the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments of the method of improving oral health and reducing tooth decay in a human subject, salivary Streptococcus mutans content is decreased in the human subject post-administration of the orally ingestible composition in accordance with the disclosure herein.

In some embodiments of the method of improving oral health and reducing tooth decay in a human subject, plaque Streptococcus mutans content is decreased in the human subject post-administration of the orally ingestible composition in accordance with the disclosure herein.

In some embodiments of the method of improving oral health and reducing tooth decay in a human subject, salivary Lactobacillus mutans content is decreased in the human subject post-administration of the orally ingestible composition in accordance with the disclosure herein.

In some embodiments of the method of improving oral health and reducing tooth decay in a human subject, plaque Lactobacillus mutans content is decreased in the human subject post-administration of the orally ingestible composition in accordance with the disclosure herein.

### BRIEF DESCRIPTION OF THE SEVERAL VIEWS OF THE DRAWINGS

The foregoing summary, as well as the following detailed description of embodiments of the compositions and methods disclosed herein, will be better understood when read in conjunction with the appended drawings of exemplary embodiments. It should be understood, however, that the invention is not limited to the precise arrangements and instrumentalities shown. The below figures illustrate examples of efficacy for compositions/methods in accordance with exemplary embodiments of the present invention.

In the drawings:

FIG. 1 describes a randomized controlled human trial on the effect of kiwifruit capsules consumptions on gut microflora in functionally constipated individuals. FIG. 1A lists a description of tested compositions and dosage amounts. FIG. 1B lists partial results of the study, underscoring the significant increase in abundance of Faecalibacterium prausnitzii in functionally constipated individuals.

FIG. 2 describes the efficacy of B. coagulans on digestion and constipation in a cohort randomly assigned to placebo vs B. coagulans Unique IS2 capsules (2×10⁹ CFU) daily for 4 weeks. FIG. 2 left panel: mean frequency of spontaneous complete bowl movement; FIG. 2 right panel: mean score of Bristol stool form. (Madempudi et al, 2019).

FIG. 3 describes a clinical study of B. coagulans in the treatment of patients with bacterial vaginosis (BV). FIG. 3A depicts the comparative analysis of BV symptoms in both groups, while FIG. 3B is a summary of the effect of B. coagulans treatment on patients suffering with BV before (day 1) and after (day 90) treatment.

FIG. 4 is an analysis of Gingival Index (GI) scores, Plaque Index (PI) scores, bleeding on probing and Glutathione peroxidase (GPx) activity in placebo (150 mg starch per tables) and treated group (10⁸ CFU B. coagulans), 3 times daily. (Jagadeesh et al, 2017).

FIG. 5 describes the effect of B. coagulans treatment on symptoms of acute diarrhea, including frequency of defecation, consistency of stool, abdominal pain, and total duration of diarrhea per day.

FIG. 6 describes the effect of B. coagulans supplementation on functional constipation in adults compared to lactulose alone and placebo. FIG. 5A shows stool frequency (left panel) and stool consistency (right panel); FIG. 5B shows defecation pain (left panel) and sensation of incomplete evacuation (right panel), and FIG. 5C shows mean abdominal pain.

FIG. 7 describes clinical trial results on the effect of heat-killed B. subtilis daily supplementation in gastrointestinal health over 6 weeks. FIG. 7A shows the number of participants showing improvement in the 3-item composite score of gas, bloating, and burping (total 76 participants); FIG. 7B depicts the odds ratio for the improvement in the 3-item composite bloating, burping, and gas score from baseline to 6 weeks compared to placebo.

FIG. 8 describes the effect of heat-killed L. plantarum (HK L-137) on immunity. FIG. 8A depicts the influence of different microorganisms (including heat-treated HK L-137) on IL-12 production. FIG. 8B depicts a diagram of lipoteichoic acid (LTA) content in HK L-37's cell wall and its effect on IL-12 production.

FIG. 9 describes the effect of heat-killed L. plantarum on immunity and quality of life. FIG. 9A shows the effect of taking heat-killed L. plantarum on T-cell growth and Th1/Th2 ratio. FIG. 9B depicts degrees of improvement in the health-related quality of life (QOL) between healthy control group subjects and healthy subjects taking heat-killed L. plantarum supplements daily. FIG. 9C shows the effect of daily intake of heat-killed L. plantarum on T-cell growth and incidence of upper respiratory tract infections (URTI). FIG. 9D depicts the effect of daily intake of heat-treated L. plantarum on immune function, inflammatory biomarkers, and lipid metabolism.

FIG. 10 describes a double-blind, placebo-controlled study on the effect of B. coagulans supplementation on plasma amino acid levels and muscle strength in resistance trained males consuming whey protein. FIG. 10A shows the effect of probiotic supplementation on muscle strength parameters; FIG. 10B shows the effect of probiotic supplementation on free amino acid levels.

FIG. 11 shows the effect of heat-treated L. plantarum supplementation on individuals suffering from chronic periodontitis.

FIG. 12 describes the effect of B. coagulans supplementation on hypercholesterolemia subjects. FIG. 12A shows the effect of B. coagulans supplementation on total serum cholesterol levels; FIG. 12B shows the effect of B. coagulans supplementation on serum triglyceride levels; FIG. 12C shows the effect of B. coagulans supplementation on serum LDL levels; FIG. 12D shows the effect of B. coagulans supplementation on serum HDL levels.

FIG. 13 summarizes results of a skin health clinical study, detailing the water content and transepidermal water loss in face and forearm of older age subjects and subjects with relatively dry skin treated with heat-killed L. plantarum (Immuno-LP20).

FIG. 14 illustrates scores for mutans streptococci strains (left-hand panels) and lactobacilli strains (right-hand panels) in salivary samples (FIG. 14A) and plaque (FIG. 14B) of children at baseline and after supplementation with B. coagulans (red columns) or placebo (blue columns).

FIG. 15 illustrates the effect of B. coagulans on irritable bowel syndrome (OBS) symptoms in adults (FIG. 15A) and in children (FIG. 15B).

### DETAILED DESCRIPTION OF THE INVENTION

Oral supplements and foods including probiotics, beneficial live microorganisms that confer health benefits on their “host” organisms (e.g., humans), have gained popularity as nutritional and health aids. However, to confer a substantial and/or lasting health benefit on those using them, the probiotics must be administered in an adequate amount and have an environment in which they can persist within the subject. It is often difficult to provide an adequate “stabilized” microbiome environment within a “host” organism to reap the full benefits of probiotic supplementation or consumption.

The interactions between probiotics, prebiotics, and postbiotics have recently garnered attention for the potential to address the issues associated with reaping the benefits of probiotics. Prebiotics may be considered to be probiotic “food”; that is, they are substrates selectively used by probiotic microorganisms in their life cycle maintenance, helping the probiotics survive during digestion to be delivered to the appropriate area of the digestive system. Prebiotics may also be used by the probiotic microorganisms to confer health benefits to the “host” organism. Most, if not, all prebiotic supplements utilize prebiotic fibers like fructooligosaccharides (FOS), xylooligosaccharides (XOS), galacto-oligosaccharides (GOS), or inulin due to low dosage requirements. However, these prebiotics are not considered FODMAP-friendly, as they are fermentable oligosaccharides which can cause unwanted GI side effects (e.g., bloating, gas).

By contrast, postbiotics may comprise inanimate or dead microorganisms, their respective parts, or waste byproducts produced by probiotic microorganisms that create a healthy microenvironment for probiotic or beneficial microorganisms to flourish. Postbiotics in the form of dead microorganisms may also confer similar health benefits to that of probiotics, but are not at risk of biological instability, since they are already non-viable. It is believed that the interplay between prebiotics, probiotics, and postbiotics may synergistically enhance the beneficial function each is known to confer separately, as the addition of pre- and postbiotics to probiotics is believed to stabilize the life cycle processes of the probiotic microorganisms.

Here, the inventors have combined pre-, pro-, and postbiotics in surprisingly efficacious manners, yielding beneficial nutritive compositions that demonstrate an enhanced, synergistic effect when consumed by a subject, leading to increased health benefits for the subject. The inventors have also developed surprisingly effective methods of using these beneficial compositions for increasing the health of subjects taking these compositions orally. Moreover, the inventors have used FODMAP-friendly prebiotics in compositions according to the disclosure herein to avoid the issues associated with typical prebiotic-containing foods and supplements.

### I. COMPOSITIONS

In an aspect of the invention, an orally ingestible nutritional composition for improving health is provided. The composition comprises an amount of gold kiwifruit powder (e.g., a prebiotic), an amount of Bacillus coagulans (a probiotic), an amount of Bacillus subtilis (a probiotic), and an amount of heat-treated Lactobacillus plantarum (postbiotic). The gold kiwifruit powder within the composition may include or consist essentially of Livaux®. The Bacillus coagulans within the composition may include or consist essentially of Unique IS-2. The heat-treated Lactobacillus plantarum within the composition may include or consist essentially of L-137. The Bacillus subtilis within the composition may include or consist essentially of BS50.

In further embodiments, an individual oral dosage form may comprise between about 75 mg and 700 mg gold kiwifruit powder; between about 5 mg and 140 mg heat-treated Lactobacillus plantarum; between about 5 mg and 90 mg Bacillus subtilis; and between about 5 mg and 90 mg Bacillus coagulans. In particular embodiments, an individual oral dosage form may comprise an amount of gold kiwifruit powder, an amount of heat-treated Lactobacillus plantarum, between about 500 million CFUs and 9 billion CFUs Bacillus coagulans, and between about 500 million CFUs and 9 billion CFUs Bacillus subtilis. In particular embodiments, the individual oral dosage form comprises an amount of gold kiwifruit powder, an amount of heat-treated Lactobacillus plantarum, about 1 billion CFUs Bacillus coagulans, and about 1 billion CFUs Bacillus subtilis. In particular embodiments, the individual oral dosage form comprises about 125 mg gold kiwifruit powder; about 25 mg heat-treated Lactobacillus plantarum; about 10 mg Bacillus subtilis; and about 10 mg Bacillus coagulans.

In further embodiments, an individual oral dosage form may comprise between about 75 mg and 100 mg gold kiwifruit powder; between about 5 mg and 20 mg heat-treated Lactobacillus plantarum; between about 5 mg and 20 mg Bacillus subtilis; and between about 5 mg and 20 mg Bacillus coagulans. In particular embodiments, the individual oral dosage form comprises about 87.5 mg gold kiwifruit powder; about 12.5 mg heat-treated Lactobacillus plantarum; about 12.5 mg Bacillus subtilis; and about 12.5 mg Bacillus coagulans.

In further embodiments, an individual oral dosage form may comprise between about 100 mg and 200 mg gold kiwifruit powder; between about 20 mg and 40 mg heat-treated Lactobacillus plantarum; between about 20 mg and 30 mg Bacillus subtilis; and between about 20 mg and 30 mg Bacillus coagulans. In particular embodiments, the individual oral dosage form comprises about 150 mg gold kiwifruit powder; about 30 mg heat-treated Lactobacillus plantarum; about 25 mg Bacillus subtilis; and about 25 mg Bacillus coagulans.

In further embodiments, an individual oral dosage form may comprise between about 200 mg and 300 mg gold kiwifruit powder; between about 40 mg and 60 mg heat-treated Lactobacillus plantarum; between about 30 mg and 40 mg Bacillus subtilis; and between about 30 mg and 40 mg Bacillus coagulans. In particular embodiments, the individual oral dosage form comprises about 250 mg gold kiwifruit powder; about 50 mg heat-treated Lactobacillus plantarum; about 35 mg Bacillus subtilis; and about 35 mg Bacillus coagulans.

In further embodiments, an individual oral dosage form may comprise between about 300 mg and 400 mg gold kiwifruit powder; between about 60 mg and 80 mg heat-treated Lactobacillus plantarum; between about 40 mg and 50 mg Bacillus subtilis; and between about 40 mg and 50 mg Bacillus coagulans. In particular embodiments, the individual oral dosage form comprises about 350 mg gold kiwifruit powder; about 70 mg heat-treated Lactobacillus plantarum; about 45 mg Bacillus subtilis; and about 45 mg Bacillus coagulans.

In further embodiments, an individual oral dosage form may comprise between about 400 mg and 500 mg gold kiwifruit powder; between about 80 mg and 100 mg heat-treated Lactobacillus plantarum; between about 50 mg and 60 mg Bacillus subtilis; and between about 50 mg and 60 mg Bacillus coagulans. In particular embodiments, the individual oral dosage form comprises about 450 mg gold kiwifruit powder; about 90 mg heat-treated Lactobacillus plantarum; about 55 mg Bacillus subtilis; and about 55 mg Bacillus coagulans.

In further embodiments, an individual oral dosage form may comprise between about 500 mg and 600 mg gold kiwifruit powder; between about 100 mg and 120 mg heat-treated Lactobacillus plantarum; between about 60 mg and 75 mg Bacillus subtilis; and between about 60 mg and 75 mg Bacillus coagulans. In particular embodiments, the individual oral dosage form comprises about 550 mg gold kiwifruit powder; about 110 mg heat-treated Lactobacillus plantarum; about 72.5 mg Bacillus subtilis; and about 72.5 mg Bacillus coagulans.

In further embodiments, an individual oral dosage form may comprise between about 600 mg and 700 mg gold kiwifruit powder; between about 120 mg and 140 mg heat-treated Lactobacillus plantarum; between about 75 mg and 90 mg Bacillus subtilis; and between about 75 mg and 90 mg Bacillus coagulans. In particular embodiments, the individual oral dosage form comprises about 650 mg gold kiwifruit powder; about 130 mg heat-treated Lactobacillus plantarum; about 87.5 mg Bacillus subtilis; and about 87.5 mg Bacillus coagulans.

According to certain embodiments of the inventive composition, the compositions are formulated such that they cause improvement in health markers within a subject consuming them (e.g., a demonstrable improvement in digestive health). Health improvements may be measured by improvement in symptoms of particular disorders (e.g., reduction in serum LDL), or increase in markers of health (e.g., increase in serum HDL).

In a further embodiment, the composition includes concentrations or amounts of the pre-, pro-, and postbiotics that are therapeutically effective to cause improvement in health markers within the subject.

In a preferred embodiment of the composition, the composition includes gold kiwifruit powder as a prebiotic. In particularly preferred embodiments, the gold kiwifruit powder includes Livaux®. Gold kiwifruit powder possesses well-demonstrated tolerability in vivo and is FODMAP-friendly. It is also shown to increase the presence of Faecalibacterium prausnitzii in the microbiome, a beneficial gut probiotic microorganism. Gold kiwifruit powder has also been shown to improve gut transit, contribute to fecal bulking, and increase mucus production in the gut.

Clinically or therapeutically effective amounts, or amounts sufficient to cause improvement in health markers within the subject, of the gold kiwifruit powder in compositions according to the invention herein include from about 75 mg and 2500 mg of gold kiwifruit powder per daily total dose, from about 75 mg and 2250 mg per daily total dose, from about 80 mg and 2000 mg, from about 95 to about 1750 mg, from about 100 to about 1500 mg, from about 110 mg to about 1250 mg, from about 120 mg to about 1000 mg, from about 125 mg to about 750 mg, from about 250 mg to about 500 mg, from about 100 to about 750 mg, from about 105 mg to about 500 mg, from about 110 to about 300 mg, from about 120 to about 200 mg, from about 125 to about 150 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg. The gold kiwifruit powder may comprise Livaux®.

In a preferred embodiment of the composition, the composition includes Bacillus coagulans as a probiotic. In particularly preferred embodiments, the B. coagulans comprises IS-2®. IS-2® has been shown to improve gut, oral, and immune health across multiple therapeutic endpoints and in different human populations.

Clinically or therapeutically effective amounts, or amounts sufficient to cause improvement in health markers within the subject, of the Bacillus coagulans in compositions according to the invention herein include from about 5 mg and 400 mg of Bacillus coagulans per daily total dose, from about 5 mg and 350 mg per daily total dose, from about 10 mg to about 300 mg, from about 15 mg to about 250 mg, from about 20 mg to about 200 mg, from about 25 mg to about 150 mg, from about 30 mg to about 100 mg, from about 35 mg to about 50 mg, from about 5 mg to about 75 mg, from about 5 mg to about 50 mg, from about 10 mg to about 40 mg, from about 10 mg to about 30 mg, from about 15 mg to about 25 mg, about 5 mg, about 10 mg, about 15 mg, about 20 mg, about 25 mg, about 30 mg, about 35 mg, about 40 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg. Clinically or therapeutically effective amounts, or amounts sufficient to cause improvement in health markers within the subject, of the Bacillus coagulans in compositions according to the invention herein include between about 500 million CFUs and 40 billion CFUs Bacillus coagulans per daily total dose, from about 500 million CFUs to about 35 billion CFUs Bacillus coagulans per daily total dose, from about 500 million CFUs to about 30 billion CFUs Bacillus coagulans per daily total dose, from about 500 million CFUs to about 25 billion CFUs Bacillus coagulans per daily total dose, from about 750 million CFUs to about 30 billion CFUs Bacillus coagulans per daily total dose, from about 750 million CFUs to about 25 billion CFUs Bacillus coagulans per daily total dose, from about 750 million CFUs to about 20 billion CFUs Bacillus coagulans per daily total dose, from about 1 billion CFUs to about 25 billion CFUs Bacillus coagulans per daily total dose, from about 1 billion CFUs to about 20 billion CFUs Bacillus coagulans per daily total dose, from about 1 billion CFUs to about 15 billion CFUs Bacillus coagulans per daily total dose, about 500 million CFUs, about 750 million CFUS, about 1 billion CFUs, about 1.25 billion CFUs, about 1.5 billion CFUS, about 1.75 billion CFUs, about 2 billion CFUs, about 2.25 billion CFUs, about 2.5 billion CFUS, about 2.75 billion CFUs, about 3 billion CFUs, about 3.25 billion CFUs, about 3.5 billion CFUS, about 3.75 billion CFUs, about 4 billion CFUs, about 4.25 billion CFUs, about 4.5 billion CFUS, about 4.75 billion CFUs, about 5 billion CFUs, about 5.25 billion CFUs, about 5.5 billion CFUS, about 5.75 billion CFUs, about 6 billion CFUs, about 7 billion CFUs, about 8 billion CFUs, about 9 billion CFUs, about 10 billion CFUs, about 15 billion CFUs, about 20 billion CFUs, about 25 billion CFUs, about 30 billion CFUs, about 35 billion CFUs, or about 40 billion CFUs. The B. coagulans may comprise IS-2®.

In a preferred embodiment of the composition, the composition includes Bacillus subtilis as a probiotic. In particularly preferred embodiments, the B. subtilis comprises OPTIBIOME® BS50™. OPTIBIOME® BS50™ has been shown to reduce bloating, burping, and flatulence to resolve problems often associated with prebiotic and/or probiotic containing products. Its effects are also found to be fast-acting.

Clinically or therapeutically effective amounts, or amounts sufficient to cause improvement in health markers within the subject, of the Bacillus subtilis in compositions according to the invention herein include from about 5 mg and 400 mg of Bacillus subtilis per daily total dose, from about 5 mg and 350 mg per daily total dose, from about 10 mg to about 300 mg, from about 15 mg to about 250 mg, from about 20 mg to about 200 mg, from about 25 mg to about 150 mg, from about 30 mg to about 100 mg, from about 35 mg to about 50 mg, from about 5 mg to about 75 mg, from about 5 mg to about 50 mg, from about 10 mg to about 40 mg, from about 10 mg to about 30 mg, from about 15 mg to about 25 mg, about 5 mg, about 10 mg, about 15 mg, about 20 mg, about 25 mg, about 30 mg, about 35 mg, about 40 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg. Clinically or therapeutically effective amounts, or amounts sufficient to cause improvement in health markers within the subject, of the Bacillus subtilis in compositions according to the invention herein include between about 500 million CFUs and 40 billion CFUs Bacillus subtilis per daily total dose, from about 500 million CFUs to about 35 billion CFUs Bacillus subtilis per daily total dose, from about 500 million CFUs to about 30 billion CFUs Bacillus subtilis per daily total dose, from about 500 million CFUs to about 25 billion CFUs Bacillus subtilis per daily total dose, from about 750 million CFUs to about 30 billion CFUs Bacillus subtilis per daily total dose, from about 750 million CFUs to about 25 billion CFUs Bacillus subtilis per daily total dose, from about 750 million CFUs to about 20 billion CFUs Bacillus subtilis per daily total dose, from about 1 billion CFUs to about 25 billion CFUs Bacillus subtilis per daily total dose, from about 1 billion CFUs to about 20 billion CFUs Bacillus subtilis per daily total dose, from about 1 billion CFUs to about 15 billion CFUs Bacillus subtilis per daily total dose, about 500 million CFUs, about 750 million CFUS, about 1 billion CFUs, about 1.25 billion CFUs, about 1.5 billion CFUS, about 1.75 billion CFUs, about 2 billion CFUs, about 2.25 billion CFUs, about 2.5 billion CFUS, about 2.75 billion CFUs, about 3 billion CFUs, about 3.25 billion CFUs, about 3.5 billion CFUS, about 3.75 billion CFUs, about 4 billion CFUs, about 4.25 billion CFUs, about 4.5 billion CFUS, about 4.75 billion CFUs, about 5 billion CFUs, about 5.25 billion CFUs, about 5.5 billion CFUS, about 5.75 billion CFUs, about 6 billion CFUs, about 7 billion CFUs, about 8 billion CFUs, about 9 billion CFUs, about 10 billion CFUs, about 15 billion CFUs, about 20 billion CFUs, about 25 billion CFUs, about 30 billion CFUs, about 35 billion CFUs, or about 40 billion CFUs. The B. subtilis may comprise OPTIBIOME® BS50™.

In a preferred embodiment of the composition, the composition includes heat-treated (i.e., killed) Lactobacillus plantarum as a postbiotic. In particularly preferred embodiments, the heat-treated L. plantarum comprises ImmunoLP20®. ImmunoLP20® has been shown to support immune health across multiple therapeutic endpoints and in different human populations.

Clinically or therapeutically effective amounts, or amounts sufficient to cause improvement in health markers within the subject, of the L. plantarum in compositions according to the invention herein include from about 5 mg and 1000 mg of L. plantarum per daily total dose, from about 5 mg and 950 mg per daily total dose, from about 5 mg to about 900 mg, from about 10 mg to about 850 mg, from about 15 mg to about 800 mg, from about 20 mg to about 750 mg, from about 25 mg to about 700 mg, from about 30 mg to about 650 mg, from about 35 mg to about 600 mg, from about 40 mg to about 550 mg, from about 45 mg to about 500 mg, from about 50 mg to about 450 mg, from about 55 mg to about 400 mg, from about 60 mg to about 350 mg, from about 65 mg to about 300 mg, from about 70 mg to about 250 mg, from about 75 mg to about 200 mg, from about 25 mg to about 75 mg, from about 30 mg to about 70 mg, from about 35 mg to about 65 mg, from about 40 mg to about 60 mg, from about 45 mg to about 55 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg. The L. plantarum may comprise ImmunoLP20®.

Compositions according to embodiments of the invention may include combinations of components each in clinically or therapeutically effective amounts. For example, in some compositional embodiments, each of the gold kiwifruit powder, the B. coagulans, the B. subtilis, and the L. plantarum may be included in therapeutically effective amounts in composition.

In some embodiments, components of the inventive composition may be combined at a w/w ratio of between about 5:1 and about 20:1 gold kiwifruit powder:Bacillus coagulans. In some embodiments, components of the inventive composition may be combined at a w/w ratio of between about 6:1 and about 18:1 gold kiwifruit powder:Bacillus coagulans. In some embodiments, components of the inventive composition may be combined at a w/w ratio of between about 8:1 and about 15:1 gold kiwifruit powder:Bacillus coagulans. In some embodiments, components of the inventive composition may be combined at a w/w ratio of between about 10:1 and about 14:1 gold kiwifruit powder:Bacillus coagulans. In some embodiments, components of the inventive composition may be combined at a w/w ratio of between about 12:1 and about 13:1 gold kiwifruit powder:Bacillus coagulans.

In some embodiments, components of the inventive composition may be combined at a w/w ratio of between about 5:1 and about 20:1 gold kiwifruit powder:Bacillus subtilis. In some embodiments, components of the inventive composition may be combined at a w/w ratio of between about 6:1 and about 18:1 gold kiwifruit powder:Bacillus subtilis. In some embodiments, components of the inventive composition may be combined at a w/w ratio of between about 8:1 and about 15:1 gold kiwifruit powder:Bacillus subtilis. In some embodiments, components of the inventive composition may be combined at a w/w ratio of between about 10:1 and about 14:1 gold kiwifruit powder:Bacillus subtilis. In some embodiments, components of the inventive composition may be combined at a w/w ratio of between about 12:1 and about 13:1 gold kiwifruit powder:Bacillus subtilis.

In some embodiments, components of the inventive composition may be combined at a w/w ratio of between about 3:1 and about 7:1 gold kiwifruit powder:Lactobacillus plantarum. In some embodiments, components of the inventive composition may be combined at a w/w ratio of between about 3.5:1 and about 6.5:1 gold kiwifruit powder:Lactobacillus plantarum. In some embodiments, components of the inventive composition may be combined at a w/w ratio of between about 4:1 and about 6:1 gold kiwifruit powder:Lactobacillus plantarum. In some embodiments, components of the inventive composition may be combined at a w/w ratio of between about 4.5:1 and about 5.5:1 gold kiwifruit powder:Lactobacillus plantarum.

In some embodiments, components of the inventive composition may be combined at a w/w ratio of between about 1.5:1 and about 3.5:1 Lactobacillus plantarum:Bacillus coagulans. In some embodiments, components of the inventive composition may be combined at a w/w ratio of between about 1.75:1 and about 3.25:1 Lactobacillus plantarum:Bacillus coagulans. In some embodiments, components of the inventive composition may be combined at a w/w ratio of between about 2:1 and about 3:1 Lactobacillus plantarum:Bacillus coagulans. In some embodiments, components of the inventive composition may be combined at a w/w ratio of between about 2.25:1 and about 2.75:1 Lactobacillus plantarum:Bacillus coagulans.

In some embodiments, components of the inventive composition may be combined at a w/w ratio of between about 1.5:1 and about 3.5:1 Lactobacillus plantarum:Bacillus subtilis. In some embodiments, components of the inventive composition may be combined at a w/w ratio of between about 1.75:1 and about 3.25:1 Lactobacillus plantarum:Bacillus subtilis. In some embodiments, components of the inventive composition may be combined at a w/w ratio of between about 2:1 and about 3:1 Lactobacillus plantarum:Bacillus subtilis. In some embodiments, components of the inventive composition may be combined at a w/w ratio of between about 2.25:1 and about 2.75:1 Lactobacillus plantarum:Bacillus subtilis.

In some embodiments, components of the inventive composition may be combined at a w/w ratio of between about 0.5:1 and about 1.5:1 Bacillus coagulans:Bacillus subtilis. In some embodiments, components of the inventive composition may be combined at a w/w ratio of between about 0.6:1 and about 1.4:1 Bacillus coagulans:Bacillus subtilis. In some embodiments, components of the inventive composition may be combined at a w/w ratio of between about 0.7:1 and about 1.3:1 Bacillus coagulans:Bacillus subtilis. In some embodiments, components of the inventive composition may be combined at a w/w ratio of between about 0.8:1 and about 1.2:1 Bacillus coagulans:Bacillus subtilis. In some embodiments, components of the inventive composition may be combined at a w/w ratio of between about 0.9:1 and about 1.1:1 Bacillus coagulans:Bacillus subtilis.

Compositions according to certain embodiments herein may be formulated as solid or liquid dosage forms. Dosage forms may be formulated for oral delivery. Oral delivery methods for compositions according to certain embodiments of the invention herein may include gummies, tablets, capsules, liquids, suspensions, chewables, soft gels, sachets, powders, syrups, liquid suspensions, emulsions, or other solutions. In particular embodiments, the orally ingestible composition is in the form of an individual dosage form. The individual dosage form may take the form of a gummy, a tablet, a capsule, or a chew. In particular embodiments, the individual dosage form is a gummy.

### II. METHODS

In an embodiment, a method of increasing the amount of Faecalibacterium prausnitzii within a digestive tract of a subject is provided. The method comprises orally administering, to the subject, an orally ingestible composition in accordance with the disclosure herein. In particular embodiments, the subject is a human. In particular embodiments, the subject is a human child under the age of 18. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve an increase in the amount of Faecalibacterium prausnitzii within the digestive tract of the subject. In some embodiments of the method of increasing the amount of Faecalibacterium prausnitzii within a digestive tract of a subject, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In an embodiment, a method of reducing constipation or relieving symptoms of constipation in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human adult. In further embodiments, the subject is an adult human female.

In some embodiments, the method of reducing constipation or relieving symptoms of constipation in a subject comprises orally delivering to the subject an effective amount or concentration of one or more of gold kiwifruit powder, Lactobacillus plantarum, Bacillus subtilis, and Bacillus coagulans to achieve a reduction in constipation or relief of symptoms of constipation in the subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day. In some embodiments of the method of reducing constipation or relieving symptoms of constipation in a subject, the subject's frequency of defecation is increased post-administration of the orally ingestible composition. In some embodiments, the subject's frequency of defecation is increased post-administration to at least three bowel movements per week.

In some embodiments of the method of reducing constipation or relieving symptoms of constipation in a subject, the subject's stools are softened and/or improved in consistency post-administration of the orally ingestible composition as measured by the Bristol stool form scale. In some embodiments, the subject's stool consistency post-administration achieves a 3 or 4 on the Bristol stool form scale.

In some embodiments of the method of reducing constipation or relieving symptoms of constipation in a subject, the subject's pain during defecation is reduced post-administration of the orally ingestible composition as measured by the constipation scoring system (CSS) scale. In some embodiments, the subject's pain during defecation is reduced post-administration to below 4 on the CSS scale. In some embodiments, the subject's pain during defecation is reduced post-administration to below 3 on the CSS scale. In some embodiments, the subject's pain during defecation is reduced post-administration to below 2 on the CSS scale. In some embodiments, the subject's pain during defecation is reduced post-administration to below 1 on the CSS scale. In some embodiments, the subject's pain during defecation is reduced post-administration to about 0 on the CSS scale.

In some embodiments of the method of reducing constipation or relieving symptoms of constipation in a subject, the subject's abdominal pain associated with constipation is reduced post-administration of the orally ingestible composition as measured by the constipation scoring system (CSS) scale. In some embodiments, the subject's abdominal pain associated with constipation is reduced post-administration to below 4 on the CSS scale. In some embodiments, the subject's abdominal pain associated with constipation is reduced post-administration to below 3 on the CSS scale. In some embodiments, the subject's abdominal pain associated with constipation is reduced post-administration to below 2 on the CSS scale. In some embodiments, the subject's abdominal pain associated with constipation is reduced post-administration to below 1 on the CSS scale. In some embodiments, the subject's abdominal pain associated with constipation is reduced post-administration to about 0 on the CSS scale.

In an embodiment, a method of improving cholesterol levels in a subject is provided. The method comprises orally administering, to the subject, an orally ingestible composition in accordance with the disclosure herein. In particular embodiments, the subject is a human. In particular embodiments, the human subject has hyperlipidemia (i.e., having serum cholesterol levels of greater than 200 mg/dL). In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve an improvement in cholesterol levels in the subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day. In some embodiments, the subject's serum HDL is increased post-administration of the orally ingestible composition. In some embodiments, the subject's serum HDL is increased post-administration between about 0.1% to about 5.0%. In some embodiments, the subject's serum HDL is increased post-administration between about 0.25% to about 4.5%. In some embodiments, the subject's serum HDL is increased post-administration between about 0.5% to about 4.0%. In some embodiments, the subject's serum HDL is increased post-administration between about 0.75% to about 3.5%. In some embodiments, the subject's serum HDL is increased post-administration between about 1.0% to about 3.0%. In some embodiments, the subject's serum HDL is increased post-administration between about 1.25% to about 2.5%. In some embodiments, the subject's serum HDL is increased post-administration by at least 0.5%, by at least 1.0%, by at least 1.5%, by at least 2.0%, by at least 2.5%, by at least 3.0%, by at least 3.5%, by at least 4.0%, or by at least 5.0%.

In some embodiments, the subject's serum LDL is decreased post-administration of the orally ingestible composition. In some embodiments, the subject's serum LDL is decreased post-administration between about 0.1% to about 5.0%. In some embodiments, the subject's serum LDL is decreased post-administration between about 0.25% to about 4.5%. In some embodiments, the subject's serum LDL is decreased post-administration between about 0.5% to about 4.0%. In some embodiments, the subject's serum LDL is decreased post-administration between about 0.75% to about 3.5%. In some embodiments, the subject's serum LDL is decreased post-administration between about 1.0% to about 3.0%. In some embodiments, the subject's serum LDL is decreased post-administration between about 1.25% to about 2.5%. In some embodiments, the subject's serum LDL is decreased post-administration by at least 0.5%, by at least 1.0%, by at least 1.5%, by at least 2.0%, by at least 2.5%, by at least 3.0%, by at least 3.5%, by at least 4.0%, or by at least 5.0%.

In some embodiments, the subject's ratio of serum HDL to serum LDL is increased post-administration between about 1.3:1: to about 1.6:1. In some embodiments, the subject's ratio of serum HDL to serum LDL is increased post-administration between about 1.35:1 to about 1.55:1. In some embodiments, the subject's ratio of serum HDL to serum LDL is increased post-administration between about 1.4:1 to about 1.5:1. In some embodiments, the subject's ratio of serum HDL to serum LDL is increased post-administration to at least about 1.35:1, to at least about 1.4:1, to at least about 1.5:1.

In some embodiments, the subject's total serum cholesterol is decreased post-administration of the orally ingestible composition. In some embodiments, the subject's total serum cholesterol is decreased post-administration between about 1.0% to about 20%. In some embodiments, the subject's total serum cholesterol is decreased post-administration between about 2.0% to about 18%. In some embodiments, the subject's total serum cholesterol is decreased post-administration between about 4.0% to about 16%. In some embodiments, the subject's total serum cholesterol is decreased post-administration between about 6.0% to about 14%. In some embodiments, the subject's total serum cholesterol is decreased post-administration between about 8.0% to about 12%. In some embodiments, the subject's total serum cholesterol is decreased post-administration by at least about 2.0%, by at least about 4.0%, by at least about 6.0%, by at least about 8.0%, by at least about 10%, by at least about 12%, by at least about 14%, by at least about 16%, by at least about 18%, by at least about 20%.

In an embodiment, a method of reducing bacterial vaginosis symptoms in a human female subject is provided. The method comprises orally administering, to the human female subject, an orally ingestible composition in accordance with the disclosure herein. In particular embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to reduce bacterial vaginosis symptoms in the subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day. In some embodiments, the human female subject is undergoing antibiotic treatment for bacterial vaginosis.

In an embodiment, a method of reducing inflammation caused by plaque-induced gingivitis in a subject is provided. The method comprises orally administering, to the subject, an orally ingestible composition in accordance with the disclosure herein. In particular embodiments, the subject is a human. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve reduction in inflammation caused by plaque-induced gingivitis in the subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day. In some embodiments, the method of reducing inflammation includes decreasing the subject's gingival index (GI) post-administration of the orally ingestible composition. In some embodiments, the subject's GI is obtained from Ram Fjord's 6 teeth (16, 21, 24, 36, 41, and 44) protocol in the FDI two-digit notation system. In some embodiments, the subject's GI is decreased post-administration by between about 2% to about 15%. In some embodiments, the subject's GI is decreased post-administration by between about 2% to about 5%. In some embodiments, the subject's GI is decreased post-administration by between about 5% to about 7%. In some embodiments, the subject's GI is decreased post-administration by between about 7% to about 9%. In some embodiments, the subject's GI is decreased post-administration by between about 9% to about 11%. In some embodiments, the subject's GI is decreased post-administration by between about 11% to about 13%. In some embodiments, the subject's GI is decreased post-administration by between about 13% to about 15%. In some embodiments, the subject's GI is decreased post-administration by at least about 2%, by at least about 5%, by at least about 7%, by at least about 9%, by at least about 11%, by at least about 13%, or by at least about 15%.

In some embodiments, the subject's bleeding on probing index (BOP) is decreased post-administration of the orally ingestible composition. In some embodiments, the subject's BOP is obtained from Ram Fjord's 6 teeth (16, 21, 24, 36, 41, and 44 in the FDI two-digit notation system). In some embodiments, the subject's BOP is decreased post-administration by between about 2% to about 15%. In some embodiments, the subject's BOP is decreased post-administration by between about 2% to about 5%. In some embodiments, the subject's BOP is decreased post-administration by between about 5% to about 7%. In some embodiments, the subject's BOP is decreased post-administration by between about 7% to about 9%. In some embodiments, the subject's BOP is decreased post-administration by between about 9% to about 11%. In some embodiments, the subject's BOP is decreased post-administration by between about 11% to about 13%. In some embodiments, the subject's BOP is decreased post-administration by between about 13% to about 15%. In some embodiments, the subject's BOP is decreased post-administration by at least about 2%, by at least about 5%, by at least about 7%, by at least about 9%, by at least about 11%, by at least about 13%, or by at least about 15%.

In some embodiments, the subject's salivary glutathione peroxidase activity is decreased post-administration of the orally ingestible composition. In some embodiments, the subject's salivary glutathione peroxidase activity is measured in terms of μg of glutathione (GSH) consumed per minute per mg of protein and measured via UV spectrophotometry. In some embodiments, the subject's salivary glutathione peroxidase activity is decreased post-administration by between about 10% to about 55%. In some embodiments, the subject's salivary glutathione peroxidase activity is decreased post-administration by between about 10% to about 15%. In some embodiments, the subject's salivary glutathione peroxidase activity is decreased post-administration by between about 15% to about 20%. In some embodiments, the subject's salivary glutathione peroxidase activity is decreased post-administration by between about 20% to about 25%. In some embodiments, the subject's salivary glutathione peroxidase activity is decreased post-administration by between about 25% to about 30%. In some embodiments, the subject's salivary glutathione peroxidase activity is decreased post-administration by between about 30% to about 35%. In some embodiments, the subject's salivary glutathione peroxidase activity is decreased post-administration by between about 35% to about 40%. In some embodiments, the subject's salivary glutathione peroxidase activity is decreased post-administration by between about 40% to about 45%. In some embodiments, the subject's salivary glutathione peroxidase activity is decreased post-administration by between about 45% to about 50%. In some embodiments, the subject's salivary glutathione peroxidase activity is decreased post-administration by between about 50% to about 55%. In some embodiments, the subject's salivary glutathione peroxidase activity is decreased post-administration by at least about 10%, by at least about 15%, by at least about 20%, by at least about 25%, by at least about 30%, by at least about 35%, by at least about 40%, by at least about 45%, by at least about 50%, or by at least about 55%.

In an embodiment, a method of reducing incidence of hepatic encephalopathy in a human subject suffering from liver cirrhosis is provided. The method comprises orally administering, to the subject, an orally ingestible composition in accordance with the disclosure herein. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve reduction in incidence of hepatic encephalopathy in the human subject suffering from liver cirrhosis. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments, the frequency of hospital admissions of the human subject suffering from liver cirrhosis is decreased in frequency post-administration of the orally ingestible composition. In some embodiments, the frequency of hospital admissions of the human subject suffering from liver cirrhosis is decreased by from about 15% to about 40%. In some embodiments, the frequency of hospital admissions of the human subject suffering from liver cirrhosis is decreased by from about 15% to about 20%. In some embodiments, the frequency of hospital admissions of the human subject suffering from liver cirrhosis is decreased by from about 20% to about 25%. In some embodiments, the frequency of hospital admissions of the human subject suffering from liver cirrhosis is decreased by from about 25% to about 30%. In some embodiments, the frequency of hospital admissions of the human subject suffering from liver cirrhosis is decreased by from about 30% to about 35%. In some embodiments, the frequency of hospital admissions of the human subject suffering from liver cirrhosis is decreased by from about 35% to about 40%.

In some embodiments, the plasma ammonia levels of the human subject suffering from liver cirrhosis are decreased post-administration of the orally ingestible composition. In some embodiments, the plasma ammonia levels of the human subject suffering from liver cirrhosis are decreased post-administration of the orally ingestible composition by from about 10% to about 30%. In some embodiments, the plasma ammonia levels of the human subject suffering from liver cirrhosis are decreased post-administration of the orally ingestible composition by from about 10% to about 15%. In some embodiments, the plasma ammonia levels of the human subject suffering from liver cirrhosis are decreased post-administration of the orally ingestible composition by from about 15% to about 20%. In some embodiments, the plasma ammonia levels of the human subject suffering from liver cirrhosis are decreased post-administration of the orally ingestible composition by from about 20% to about 25%. In some embodiments, the plasma ammonia levels of the human subject suffering from liver cirrhosis are decreased post-administration of the orally ingestible composition by from about 25% to about 30%.

In an embodiment, a method of improving digestive health in a human subject suffering from acute diarrhea is provided. The method comprises orally administering, to the subject, an orally ingestible composition in accordance with the disclosure herein. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve improvement in digestive health in a human subject suffering from acute diarrhea. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 500 million CFUs and 40 billion CFUs Bacillus subtilis per day. In some embodiments, the subject receives, via oral administration of compositions in accordance with the disclosure herein, between about 5 mg and 400 mg of Bacillus coagulans per day. In some embodiments, the subject receives, via oral administration of compositions in accordance with the disclosure herein, between about 500 million CFUs and 40 billion CFUs Bacillus coagulans per day.

In some embodiments, the incidence frequency of diarrhea and/or associated symptoms is reduced in the human subject suffering from acute diarrhea post-administration of the orally ingestible composition. In some embodiments, the total duration of diarrhea is reduced in the human subject suffering from acute diarrhea post-administration of the orally ingestible composition by about 2 min to about 15 min. In some embodiments, the total duration of diarrhea is reduced in the human subject suffering from acute diarrhea post-administration of the orally ingestible composition by about 2 min to about 5 min. In some embodiments, the total duration of diarrhea is reduced in the human subject suffering from acute diarrhea post-administration of the orally ingestible composition by about 5 min to about 7 min. In some embodiments, the total duration of diarrhea is reduced in the human subject suffering from acute diarrhea post-administration of the orally ingestible composition by about 7 min to about 9 min. In some embodiments, the total duration of diarrhea is reduced in the human subject suffering from acute diarrhea post-administration of the orally ingestible composition by about 9 min to about 11 min. In some embodiments, the total duration of diarrhea is reduced in the human subject suffering from acute diarrhea post-administration of the orally ingestible composition by about 11 min to about 13 min. In some embodiments, the total duration of diarrhea is reduced in the human subject suffering from acute diarrhea post-administration of the orally ingestible composition by about 13 min to about 15 min. In some embodiments, the total duration of diarrhea is reduced in the human subject suffering from acute diarrhea post-administration of the orally ingestible composition by at least about 2 min, by at least about 5 min, by at least about 7 min, by at least about 9 min, by at least about 11 min, by at least about 13 min, or by at least about 15 min.

In some embodiments, frequency of defecation of the human subject suffering from acute diarrhea is reduced post-administration of the orally ingestible composition by from about 9 per day to about 1 per day. In some embodiments, frequency of defecation of the human subject suffering from acute diarrhea is reduced post-administration of the orally ingestible composition by from about 9 per day to about 8 per day. In some embodiments, frequency of defecation of the human subject suffering from acute diarrhea is reduced post-administration of the orally ingestible composition by from about 8 per day to about 7 per day. In some embodiments, frequency of defecation of the human subject suffering from acute diarrhea is reduced post-administration of the orally ingestible composition by from about 7 per day to about 6 per day. In some embodiments, frequency of defecation of the human subject suffering from acute diarrhea is reduced post-administration of the orally ingestible composition by from about 6 per day to about 5 per day. In some embodiments, frequency of defecation of the human subject suffering from acute diarrhea is reduced post-administration of the orally ingestible composition by from about 5 per day to about 4 per day. In some embodiments, frequency of defecation of the human subject suffering from acute diarrhea is reduced post-administration of the orally ingestible composition by from about 4 per day to about 3 per day. In some embodiments, frequency of defecation of the human subject suffering from acute diarrhea is reduced post-administration of the orally ingestible composition by from about 3 per day to about 2 per day. In some embodiments, frequency of defecation of the human subject suffering from acute diarrhea is reduced post-administration of the orally ingestible composition by from about 2 per day to about 1 per day. In some embodiments, frequency of defecation of the human subject suffering from acute diarrhea is reduced post-administration of the orally ingestible composition by at least about 1 per day, at least about 2 per day, at least about 3 per day, at least about 4 per day, at least about 5 per day, at least about 6 per day, at least about 7 per day, at least about 8 per day, or at least about 9 per day.

In some embodiments, abdominal pain of the human subject suffering from acute diarrhea is reduced post-administration of the orally ingestible composition. In some embodiments, abdominal pain of the human subject suffering from acute diarrhea is reduced post-administration of the orally ingestible composition by about 4 (severe) to about 0 (absent). In some embodiments, abdominal pain of the human subject suffering from acute diarrhea is reduced post-administration of the orally ingestible composition by about 4 (severe) to about 3. In some embodiments, abdominal pain of the human subject suffering from acute diarrhea is reduced post-administration of the orally ingestible composition by about 3 to about 2. In some embodiments, abdominal pain of the human subject suffering from acute diarrhea is reduced post-administration of the orally ingestible composition by about 2 to about 1. In some embodiments, abdominal pain of the human subject suffering from acute diarrhea is reduced post-administration of the orally ingestible composition by about 1 to about 0 (absent).

In some embodiment, stool consistency of the human subject suffering from acute diarrhea is ameliorated post-administration of the orally ingestible composition. In some embodiments, stool consistency of the human subject suffering from acute diarrhea is ameliorated post-administration of the orally ingestible composition by from about 4 (loose consistency) to about 1 (hard consistency). In some embodiment, stool consistency of the human subject suffering from acute diarrhea is ameliorated post-administration of the orally ingestible composition by from about 4 (loose consistency) to about 3. In some embodiment, stool consistency of the human subject suffering from acute diarrhea is ameliorated post-administration of the orally ingestible composition by from about 3 to about 2. In some embodiment, stool consistency of the human subject suffering from acute diarrhea is ameliorated post-administration of the orally ingestible composition by from about 2 to about 1 (hard consistency).

In an embodiment, a method of alleviating irritable bowel syndrome (IBS) symptoms in a human subject suffering from IBS is provided. The method comprises orally administering, to the subject, an orally ingestible composition in accordance with the disclosure herein. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve alleviation of IBS symptoms in the human subject suffering from IBS. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 5 mg and 400 mg of Bacillus coagulans per day.

In some embodiments, the abdominal pain and/or discomfort of the human subject suffering from IBS is reduced post-administration of the orally ingestible composition.

In some embodiments, the incidence frequency of complete spontaneous bowel movements is increased post-administration in the human subject suffering from IBS.

In some embodiments, the abdominal bloating of the human subject suffering from IBS is reduced post-administration of the orally ingestible composition.

In an embodiment, a method of reducing burping in a human subject is provided. The method comprises orally administering, to the subject, an orally ingestible composition in accordance with the disclosure herein. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve reduction in burping of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 5 mg and 400 mg of Bacillus coagulans per day.

In an embodiment, a method of reducing flatulence in a human subject is provided. The method comprises orally administering, to the subject, an orally ingestible composition in accordance with the disclosure herein. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to achieve reduction in flatulence of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 5 mg and 400 mg of Bacillus coagulans per day.

In an embodiment, a method of supporting protein digestion in a human subject is provided. The method comprises orally administering, to the subject, an orally ingestible composition in accordance with the disclosure herein. In particular embodiments, the subject is a human adult. In further embodiments, the subject is an adult human female. In further embodiments, the subject is an adult human male. In some embodiments, the method comprises orally delivering to the subject an effective amount or concentration of one or more of gold kiwifruit powder, Lactobacillus plantarum, Bacillus subtilis, and Bacillus coagulans to support protein digestion in the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 5 mg and 400 mg of Bacillus coagulans per day.

In an embodiment, a method of improving the immune response of a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In some embodiments, the method comprises orally delivering to the subject an effective amount or concentration of one or more of gold kiwifruit powder, Lactobacillus plantarum, Bacillus subtilis, and Bacillus coagulans to improve the immune response of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 5 mg and 400 mg of Bacillus coagulans per day.

In some embodiments, the serum IL-12 is increased in the human subject post-administration of the orally ingestible composition.

In some embodiments, the serum IFN-β is increased in the human subject post-administration of the orally ingestible composition.

In some embodiments, T-cell activity is increased in the human subject post-administration of the orally ingestible composition.

In some embodiments, incidence frequency of upper respiratory tract infections is decreased in the human subject post-administration of the orally ingestible composition.

In some embodiments, hepatic inflammation is decreased in the human subject post-administration of the orally ingestible composition.

In some embodiments, serum aspartate transaminase is decreased in the human subject post-administration of the orally ingestible composition.

In some embodiments, serum alanine transaminase is decreased in the human subject post-administration of the orally ingestible composition.

In some embodiments, seasonal increase in total cholesterol in the human subject is suppressed post-administration of the orally ingestible composition.

In some embodiments, seasonal increase in serum LDL in the human subject is suppressed post-administration of the orally ingestible composition.

In some embodiments, activation of immune cells in the digestive tract of the human subject is increased post-administration of the orally ingestible composition.

In some embodiments, the Th1:Th2 ratio is increased in the human subject post-administration of the orally ingestible composition.

In some embodiments, the incidence frequency of colds and cold-like illness in the human subject is decreased post-administration of the orally ingestible composition.

In some embodiments, the incidence frequency of influenza and influenza-like illness in the human subject is decreased post-administration of the orally ingestible composition.

In an embodiment, a method of improving skin health in a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In particular embodiments, the human subject is suffering from dry skin. In some embodiments, the method comprises orally delivering to the subject an effective amount or concentration of one or more of gold kiwifruit powder, Lactobacillus plantarum, Bacillus subtilis, and Bacillus coagulans to improve the skin health of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 5 mg and 400 mg of Bacillus coagulans per day.

In some embodiments, water content within skin of the human subject experiences a greater increase compared to placebo post-administration of the orally ingestible composition. In some embodiments, the skin of the human subject is non-facial body skin. In particular embodiments, the body skin is forearm skin.

In some embodiments, transepidermal water loss from skin of the human subject has a greater reduction compared to placebo post-administration of the orally ingestible composition. In some embodiments, the skin of the human subject is facial skin.

In an embodiment, a method of improving chronic periodontitis symptoms in a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In particular embodiments, the human subject is suffering from chronic periodontitis. In some embodiments, the method comprises orally delivering to the subject an effective amount or concentration of one or more of gold kiwifruit powder, Lactobacillus plantarum, Bacillus subtilis, and Bacillus coagulans to improve the chronic periodontitis symptoms of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 5 mg and 400 mg of Bacillus coagulans per day.

In an embodiment, a method of improving protein and amino acid absorption in a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In further embodiments, the human subject is an adult human male. In some embodiments, the method comprises orally delivering to the subject an effective amount or concentration of one or more of gold kiwifruit powder, Lactobacillus plantarum, Bacillus subtilis, and Bacillus coagulans to improve the protein and amino acid absorption of the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 5 mg and 400 mg of Bacillus coagulans per day.

In some embodiments, plasma amino acids in the human subject are increased post-administration of the orally ingestible composition in accordance with the disclosure herein. In particular embodiments, the increased plasma amino acids comprise branched-chain amino acids.

In some embodiments, the method of improving protein and amino acid absorption further comprises orally administering a protein-containing food to the human subject. In some embodiments, the protein-containing food comprises whey protein. In particular embodiments, muscle strength of the human subject has a greater increase post-administration of the orally ingestible composition and the protein-containing food in comparison to muscle strength of human subjects consuming only the protein-containing food.

In an embodiment, a method of improving oral health and reducing tooth decay in a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In further embodiments, the human subject is an adult human male. In some embodiments, the method comprises orally delivering to the subject an effective amount or concentration of one or more of gold kiwifruit powder, Lactobacillus plantarum, Bacillus subtilis, and Bacillus coagulans to improve the oral health of and reduce tooth decay in the human subject. In some embodiments, the subject receives, via oral administration of compositions in accordance with the disclosure herein, between about 75 mg and 2500 mg of gold kiwifruit powder per day. In some embodiments, the subject receives, via oral administration of compositions in accordance with the disclosure herein, between about 5 mg and 1000 mg of heat-treated Lactobacillus plantarum per day. In some embodiments, the subject receives, via oral administration of compositions in accordance with the disclosure herein, between about 5 mg and 400 mg of Bacillus subtilis per day. In some embodiments, the subject receives, via oral administration of compositions in accordance with the disclosure herein, between about 5 mg and 400 mg of Bacillus coagulans per day.

In some embodiments, salivary Streptococcus mutans content is decreased in the human subject post-administration of the orally ingestible composition in accordance with the disclosure herein.

In some embodiments, plaque Streptococcus mutans content is decreased in the human subject post-administration of the orally ingestible composition in accordance with the disclosure herein.

In some embodiments, salivary Lactobacillus mutans content is decreased in the human subject post-administration of the orally ingestible composition in accordance with the disclosure herein.

In some embodiments, plaque Lactobacillus mutans content is decreased in the human subject post-administration of the orally ingestible composition in accordance with the disclosure herein.

### III. EXAMPLES

### Example 1: Effect of Kiwifruit Capsules Consumptions on Gut Microflora in Functionally Constipated Individuals

A randomized controlled human trial demonstrated that consumption of gold kiwifruit capsules (600 mg/capsule, up to 2400 mg/day for 14 days vs. isomalt placebo) significantly increased Faecalibacterium bacterial group abundance in functionally constipated subjects. Blatchford et al., (2017) Consumption of kiwifruit capsules increases Faecalibacterium prausnitzii abundance in functionally constipated individuals: a randomised controlled human trial, Journal of Nutritional Science (2017), vol. 6, e52, page 1 of 10 (herein incorporated by reference in its entirety). The study design was a randomised double-blind placebo-controlled cross-over trial with participants consuming four different interventions for 4 weeks each, with a 2-week washout between each intervention. The interventions were delivered in 4×600 mg capsules supplied by Anagenix Ltd prepared to look identical to preserve intervention blinding.

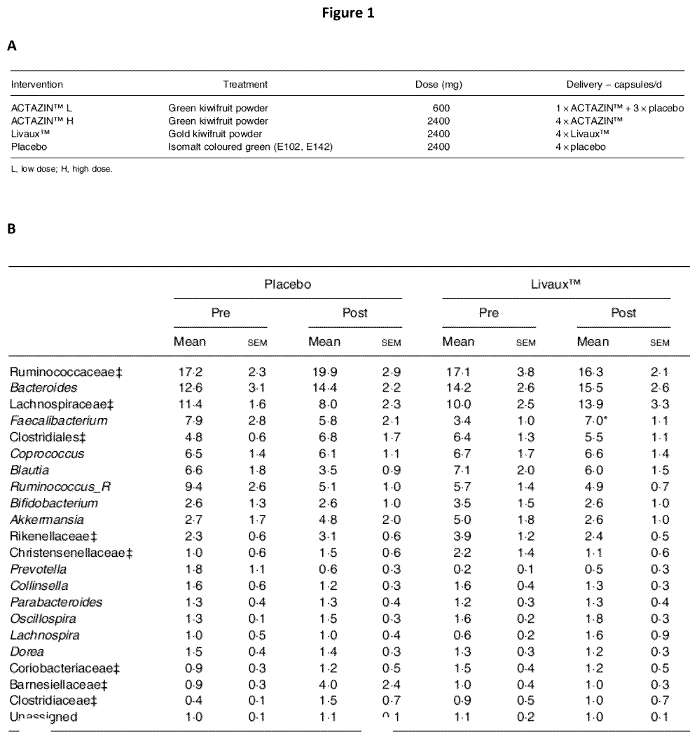

Participants consumed four different intervention combinations: placebo (isomalt coloured green) (2400 mg/d), ACTAZIN™ L (600 mg/d), ACTAZIN™ H (2400 mg/d) and Livaux™ (2400 mg/d) for 28 d each intervention, with a 14 d washout period between each treatment phase. FIG. 1A. ACTAZIN™ L (low dose, green kiwifruit) and ACTAZIN™ H (high dose, green kiwifruit) were formulated from cold-processed Actinidia chinensis var. deliciosa ‘Hayward’ green kiwifruit and Livaux™ was formulated from cold-processed Actinidia chinensis var. chinensis ‘Zesy002’ gold-fleshed kiwifruit. The placebo ingredient was isomalt (1-O-α-Dglucopyranosyl-D-mannitol). Subjects were asked to exclude high-fibre dietary supplements such as Metamucil, Benefibre and Phloe as well as maintaining their habitual dietary intakes and physical activity habits and to refrain from eating fresh kiwifruit for the study period. At the beginning and end of each 4-week intervention period, participants were asked to provide a faecal sample. The washout period of 2 weeks was chosen to allow sufficient time to return bowel habits to baseline for the parameters measured (microbial ecology, microbial metabolites and SmartPill® pH measurements).

This study investigated the impact of ACTAZIN™ green (2400 and 600 mg) and Livaux™ (2400 mg) gold kiwifruit supplements on faecal microbial composition and metabolites in healthy and functionally constipated (FC) participants. The participants were recruited into the healthy group (n 20; one of whom did not complete the study) and the FC group (n 9), each of whom consumed all the treatments and a placebo (isomalt) for 4 weeks in a randomised cross-over design interspersed with 2-week washout periods. Modification of faecal microbiota composition and metabolism was determined by 16S rRNA gene sequencing and GC, and colonic pH was calculated using SmartPill® wireless motility capsules. A total of thirty-two taxa were measured at greater than 1% abundance in at least one sample, ten of which differed significantly between the baseline healthy and FC groups. Specifically, Bacteroidales and Roseburia spp. were significantly more abundant (P<0.05) in the healthy group and taxa including Ruminococcaceae, Dorea spp. and Akkermansia spp. were significantly more abundant (P<0.05) in the FC group. In the FC group, Faecalibacterium prausnitzii abundance significantly increased (P=0.024) from 3.4 to 7.0% following Livaux™ supplementation, with eight of the nine participants showing a net increase. Lower proportions of F. prausnitzii are often associated with gastrointestinal disorders. The discovery that Livaux™ supplementation increased F. prausnitzii abundance offers a potential strategy for improving gut microbiota composition, as F. prausnitzii is a butyrate producer and has also been shown to exert anti-inflammatory effects in many studies.

### Example 2: Effect of Bacillus coagulans Unique IS2 in Constipation

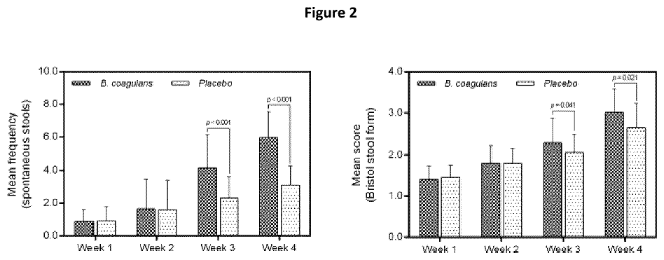

Functional constipation has a high prevalence in both adults and children affecting quality of life. Evidence suggests that probiotics can reduce the symptoms of constipation. As the effects of probiotics are strain specific, the efficacy of Bacillus coagulans Unique IS2 in the treatment of functional constipation in adults was evaluated. Subjects (n=100) diagnosed with functional constipation were supplemented with either B. coagulans Unique IS2 (2 billion CFU) or placebo capsules once daily for 4 weeks. Subjects were evaluated for treatment success (defined as three or more spontaneous stools per week), stool consistency, difficulty of defecation, defecation and abdominal pain. By the end of the fourth week, there was a statistically significant (p<0.001) increase in number of bowel movements in the probiotic treated group as compared to placebo (FIG. 2). Ninety eight percent of subjects in the probiotic group achieved normal stool consistency as compared to placebo (74%). Moreover, there was relief from symptoms of incomplete evacuation, painful defecation and abdominal pain associated with constipation in probiotic treated group as compared to placebo. In conclusion, B. coagulans Unique IS2 significantly decreased the symptoms of constipation indicating effectiveness of the strain in the treatment of constipation.

### Example 3: Effect of Bacillus coagulans Unique IS-2 in the Treatment of Patients with Bacterial Vaginosis

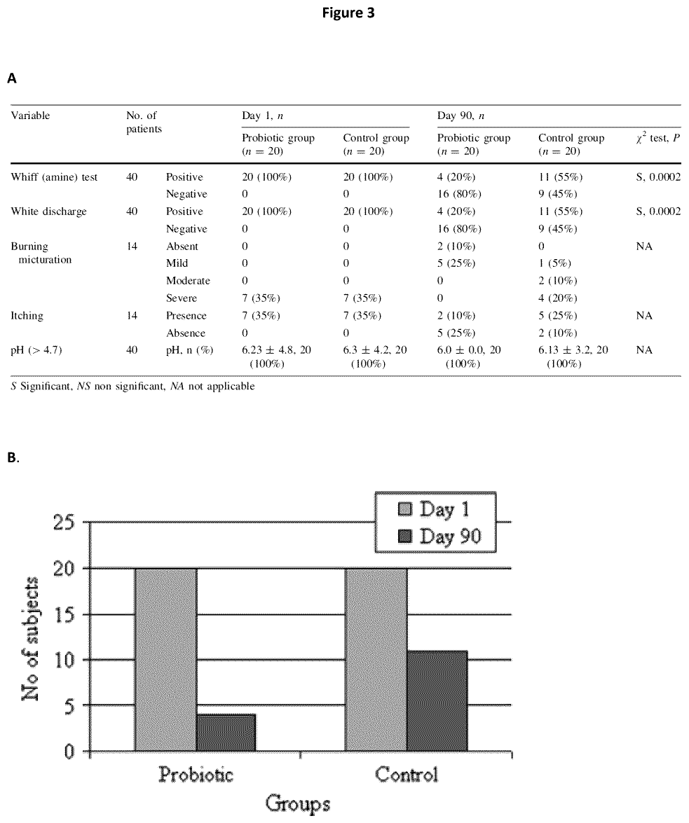

Control group was given standard vaginosis treatment alone (Ofloxacin-Ornidazole 200-500 mg/capsule/day for 5 days along with co-kimaxazol vaginal peccaries for 3 days), as the test group took standard vaginosis treatment plus 2 capsules of B. coagulans (10⁹ CFU/capsule) twice a day for 90 days (see FIG. 3), from Sudha et al., 2011, Clinical Study of Bacillus coagulans Unique IS-2 (ATCC PTA-11748) in the Treatment of Patients with Bacterial Vaginosis, Indian J Microbiol (July-September 2012) 52(3):396-399 397, herein incorporated by reference in its entirety.

Forty women reported at out-patient department in Government Medical College and Hospital Aurangabad, India, with BV were examined. The study was approved by the Institutional Ethics Committee (ref no: ICE/GMCA/114/2008) and all subjects gave written informed consent before any research activity was initiated. The inclusion criteria for the subjects was based on presence of the following symptoms such as, white discharge, positive for whiff test, vaginal pH greater than 4.7 (normal pH between 3.8 and 4.5), along with other symptoms like, burning micturation, itching, soreness and redness at vulva.

Participants fulfilling the entry criteria were divided equally (control 20 and probiotic 20). The following observation are noted for age (control group, 33±3 years and probiotic group, 32.5±3), history of previous vaginosis (control group, 75% or 15/20 and probiotic group, 75% or 15/20) and severity of current vaginosis infection (burning micturation and itching, 35% in each group). Study subjects were not blinded to the treatment they received. The control group patients were given standard vaginosis treatment alone (Ofloxacin-Ornidazole with a strength of 200-500 mg per capsule/day for 5 days along with co-kimaxazol vaginal peccaries for 3 days). The probiotic group patients took standard vaginosis treatment plus two capsules of B. coagulans Unique IS-2 (1×9 10⁹ CFU/capsule) twice a day before meals for 90 days. At the end of the treatment the 80% of probiotic group subjects showed significant positive response as revealed by reduction of vaginosis symptoms compared to the control group which exhibited reduction in 45% subjects only. Thus, the results of present study indicate that strain Bacillus coagulans Unique IS-2 can provide benefits to women being treated with antibiotics to cure an infectious condition.

### Example 4: Effect of Bacillus coagulans on Plaque Induced Gingivitis

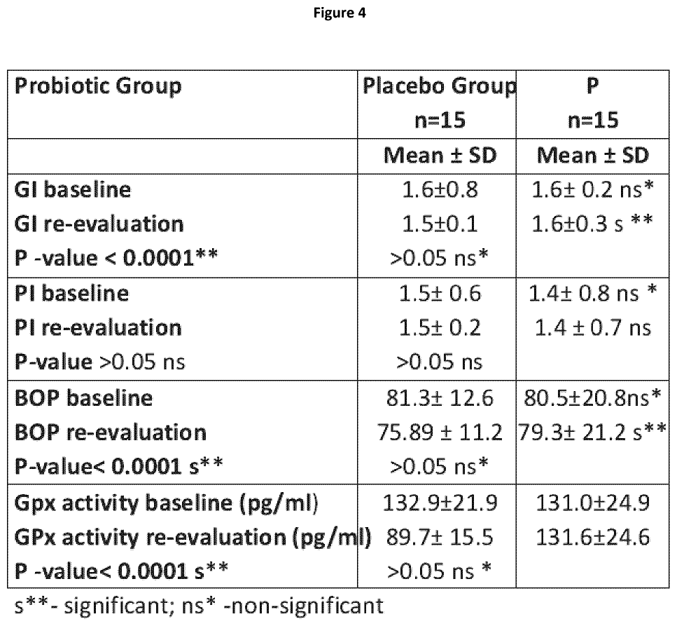

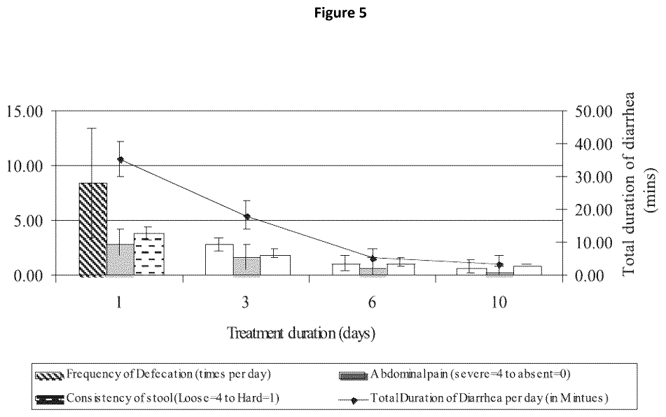

Thirty subjects with plaque induced gingivitis were enrolled in the study. At baseline, gingival index (GI) and plaque index (PI) were assessed. Saliva samples were collected for glutathione peroxidase (GPx) activity analysis and to determine load of lactobacilli. Subsequently, participants were randomly provided with chewable tablets to be consumed 3 times daily for 3 months containing 100 million CFU/tablet of B. coagulans or without B. coagulans (placebo). After 3 months, recording of GI, PI, and saliva sampling were repeated. At baseline, mean GI, and mean PI did not differ significantly between groups. At re-evaluation, mean GI, and bleeding on probing of the probiotic group were both significantly lower (p<0.0001) than in the placebo group. Mean PI level did not differ significantly between the groups. In the probiotic group, mean GPx activity was significantly lower (p<0.02) than in the placebo group at re-evaluation (see FIG. 4), from Jagadeesh et al., Clinical effect of pro-biotic containing Bacillus coagulans on plaque induced gingivitis: a randomised clinical pilot study, NUJHS Vol. 7, No. 3, September 2017, herein incorporated by reference in its entirety.

### Example 5: Effect of Bacillus coagulans Strain Unique IS-2 in the Treatment of Patients with Acute Diarrhea

The objective of this study was to evaluate the efficacy and safety of Bacillus coagulans strain Unique IS-2 in the treatment of patients with acute diarrhea. To this end, a total of 28 patients with acute diarrhea were included in a prospective, phase II clinical study upon obtaining consent and ethical committee approval. The trial was performed on patients of both sexes between 18 to 55 years of age and having grade 3 loose motions in last 24 hours for less than 7 Days. All patients were assigned to receive capsule (containing 2 billion CFU of Bacillus coagulans strain Unique IS-2) two times for a duration of 10 days. Efficacy was evaluated by assessment of duration of diarrhea (in minutes) and frequency of defecation (times per day), abdominal pain (3-severe; 2-moderate; 1-mild; 0=absent) and consistency of stool (1=normal, 2=loose, 3=semi liquid, and 4=liquid). Safety aspects of capsule were evaluated by assessment of incidence, type of adverse events, physical examination, and clinical laboratory test values (CBC, SGPT, serum creatinine, stool routine and microscopy). Concomitant medications including rescue medications were monitored throughout the study. Efficacy assessment for duration of diarrhea, frequency of defecation, abdominal pain, consistency of stool and fever was done on 1, 3, 6 and 10 days. Mean values for duration of diarrhea decreased from 35.60±5.46 to 3.52±2.69 min per day; frequency of defecation was decreased from 7.96±3.89 to 0.76±0.60 times per day; abdominal pain decreased from 3.16±0.99 to 0.36±0.49; and consistency of stool improved from 3.84±0.55 to 1.00±0.00. No significant change in safety parameters was observed during treatment. This trial demonstrates that utilization of B. coagulans Unique IS-2 strain is efficient and safe to treat the patients with acute diarrhea (see FIG. 5), from Sudha and Bhonagri, 2011, Efficacy Of Bacillus coagulans Strain Unique Is-2 In The Treatment Of Patients With Acute Diarrhea, International Journal of Probiotics and Prebiotics Vol. 7, No. 1, 2012, herein incorporated by reference in its entirety.

### Example 6: Effect of Bacillus coagulans Unique IS-2 with Lactulose on Functional Constipation in Adults

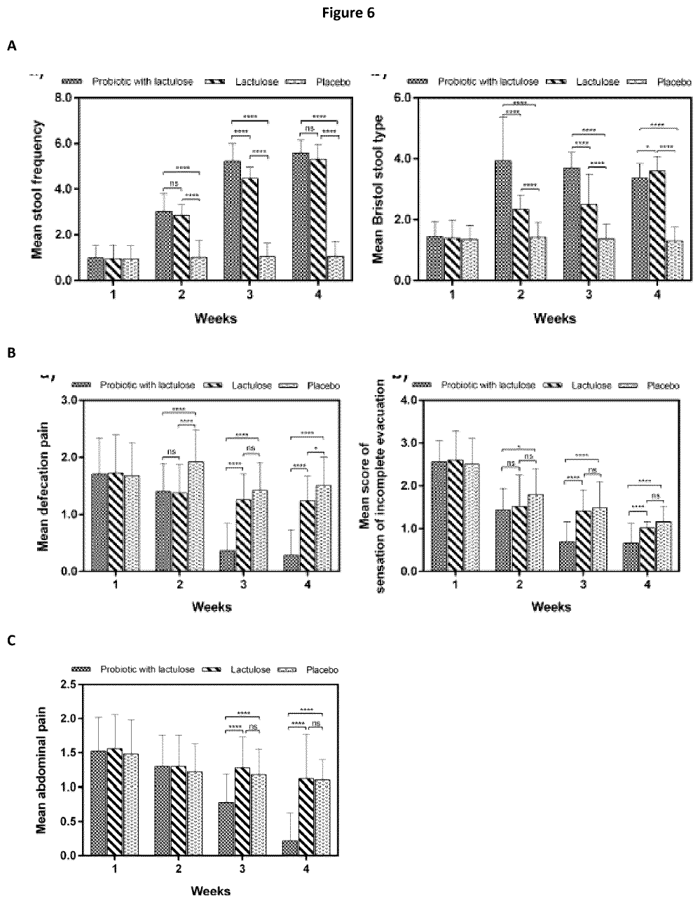

This double-blind randomized study evaluated the efficacy of a combination of Bacillus coagulans Unique IS-2 and lactulose in the treatment of functional constipation in adults. In it, 150 participants diagnosed with functional constipation (Rome III criteria) were randomised (1:1:1) and supplemented daily with 15 mL suspension of probiotic (B. coagulans Unique IS-2, 2×109 spores) with lactulose (10 g) (group 1) or lactulose (10 g) (group 2) or placebo (water) (group 3) for 4 weeks. The primary (stool frequency) and secondary outcome measures (stool consistency, sensation of incomplete evacuation, defecation- and abdominal pain) were recorded weekly for up to 4 weeks. Bacillus coagulans Unique IS-2 with lactulose showed significant changes in stool frequency as compared to lactulose treatment alone; however, at the end of the trial, it was found insignificant due to the gradual increase of stool frequency score of lactulose treatment. The changes observed in stool consistency were early (2nd week) and remained consistent up to end of the trial. The significant reduction of sensation of incomplete evacuation, defecation pain, and abdominal pain correlated with the strain's ability to produce short-chain fatty acids. No adverse events were observed in any of the groups, and all the vital parameters were normal during the course of the study. Overall, results indicated that the addition of B. coagulans Unique IS-2 to lactulose reduced time required to relieve constipation as compared to lactulose alone. In conclusion, B. coagulans Unique IS-2 with lactulose is more effective than lactulose alone to relieve symptoms of constipation in a shorter period. (see FIG. 6), from Venkataraman et al., Effect of Bacillus coagulans Unique IS2 with Lactulose on Functional Constipation in Adults: a Double-Blind Placebo Controlled Study, Probiotics and Antimicrobial Proteins https://doi.org/10.1007/s12602-021-09855-8, herein incorporated by reference in its entirety.

### Example 7: Effect of Bacillus subtilis BS350 on Gastrointestinal Symptoms in Adults

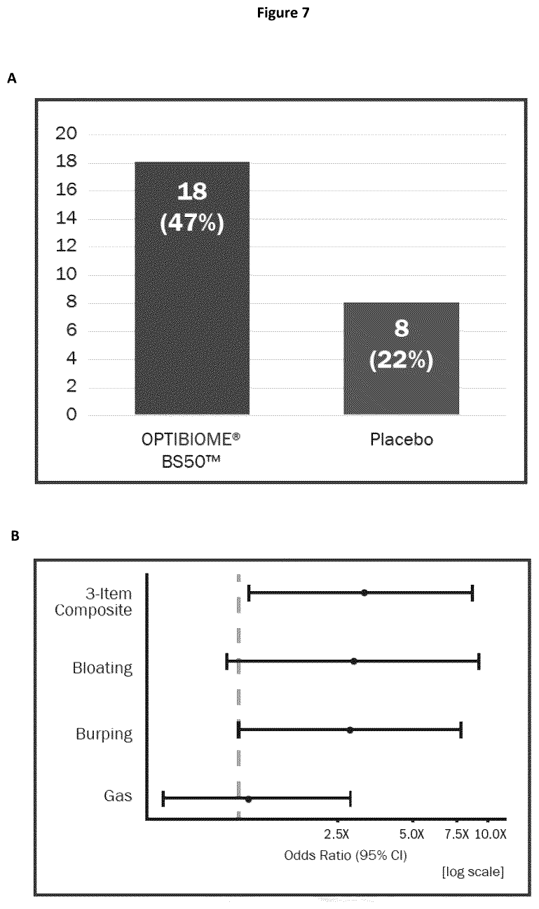

Safety and efficacy of daily supplementation of Bacillus subtilis BS50 was evaluated for 6 weeks in a randomized, double-blind, placebo-controlled, parallel clinical trial of 76 healthy adults. Each probiotic capsule contained 2×10⁹ CFU of B. subtilis BS50 with identity-preserved maltodextrin extracted from waxy maize as the excipient. The placebo capsule only contained identity-preserved maltodextrin. Before and during supplementation, gastrointestinal symptoms were recorded daily using a multi-symptom questionnaire. Clinical chemistry, hematology, plasma lipids, and intestinal permeability and inflammation markers were measured at baseline and end of study. Compared to placebo, 2×10⁹ colony-forming units (CFU) BS50 per day increased the proportion of participants showing improvement from baseline to week 6 in the composite score for bloating, burping, and flatulence (47.4% vs. 22.2%), whereby the odds of detecting an improvement were higher with BS50 (OR [95% CI]: 3.2 [1.1, 8.7], p=0.024). Analyses of individual gastrointestinal symptoms indicate that BS50 increased the proportion of participants showing an improvement at week 6 compared to placebo for burping (44.7% vs. 22.2%, p=0.041) and bloating (31.6% vs. 13.9%, p=0.071), without affecting other symptoms. There were no clinically meaningful changes in clinical chemistry, hematology, plasma lipids and intestinal permeability and other inflammation markers. In conclusion, the results suggest that dietary supplementation of 2×10⁹ CFU Bacillus subtilis BS50 per day is a well-tolerated and safe strategy to alleviate gas-related gastrointestinal symptoms in healthy adults (FIG. 7).

### Example 8: Effect of Heat-Killed Lactobacillus Plantarum L-137 Strain (HK L-137) on Immunity and Quality of Life

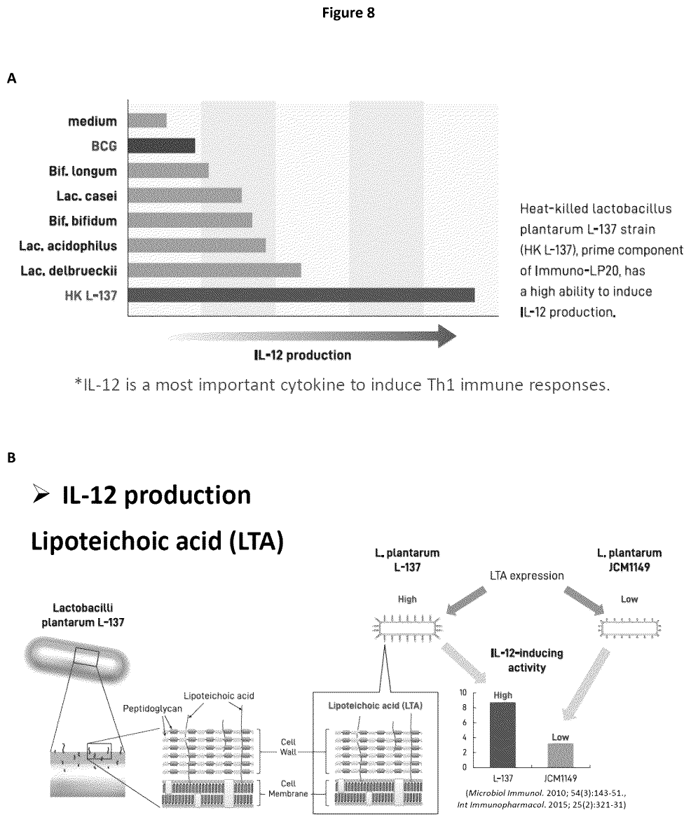

The postbiotic HK L-137 is found in foods like pickles and dairy, as well as those produced by Southeast Asian fermentation methods, and is identified as an immunobiotic because it promotes immunity (House Wellness Foods Corporation Immuno-LP20 white paper, incorporated herein in its entirety). The Immuno-LP20 supplement consists of 80% dextrin and 20% heat-killed L. plantarum L-137. The described clinical studies used 10 mg of HK L-137 in a total of 50 mg Immuno-LP20 supplement and showed that Immuno-LP20 supplementation increased IL-12 production significantly more than other lactic bacteria strains (FIG. 8A). According to FIG. 7B's depictions, the higher lipoteichoic acid (LTA) content in HK L-37's cell wall is responsible for its ability to induce IL-12. Additionally, FIG. 8B shows that L-37 induces a much higher IL-12 production than other lactic acid bacteria.

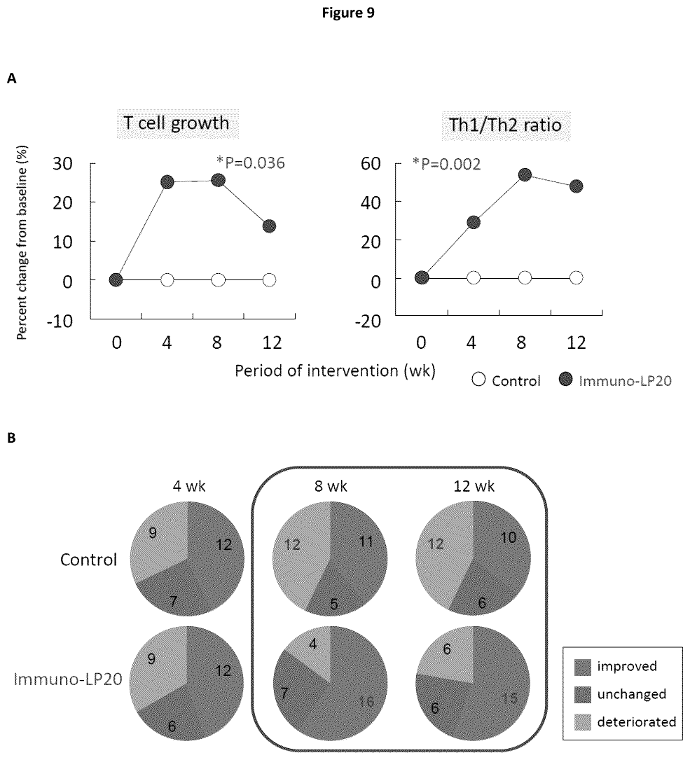

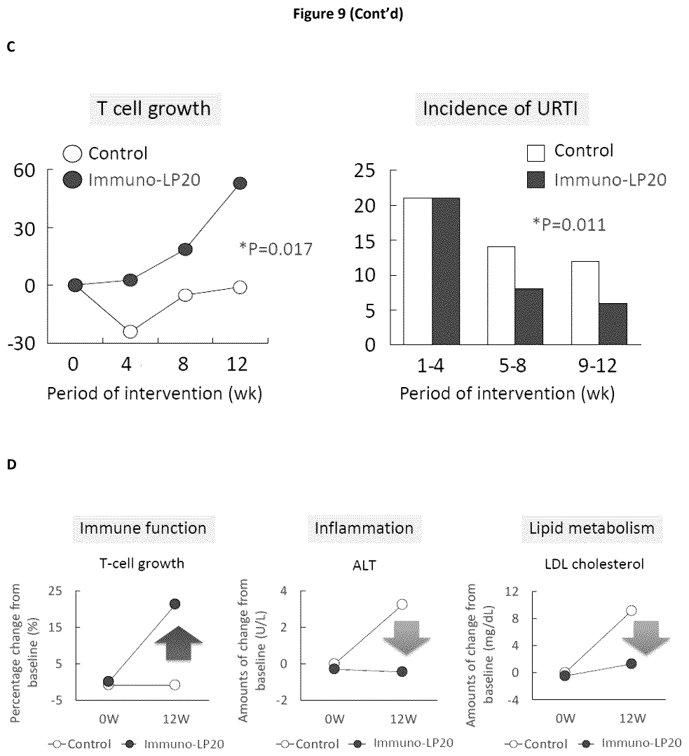

Over a 12-week period of daily supplementation, Immuno-LP20 significantly increased T-cell growth. Those taking heat-killed L. plantarum supplements daily sustained increases in Th1-related immune functions, such as T cell growth and Th1/Th2 ratio from 4 week after the start of dietary intervention (FIG. 9A, left panel) and Th1 to Th2 immune response ratio (FIG. 9A, right panel) in healthy subjects from 4 weeks after the start of dietary intervention. This correlated with higher health-related quality of life (QOL) in the Immuno-LP20 group compared to the placebo group 8 weeks after the start of the dietary intervention\. The degrees of improvement in the health-related quality of life (QOL) were higher from 8 week in the Immuno-LP group that in the control group (FIG. 8B). Taken from J. Nutr. 2006; 136(12):3069 73, herein incorporated by reference in its entirety. FIG. 9D: Daily intake of Immuno-LP20 augmented immune function and suppressed elevated serum biomarkers of inflammation and lipid metabolism in healthy subjects with a moderately high BMI (23-29.9 kg/m2).

In a second clinical study (Cohen et al., N Engl J Med, 325, 606-612 (1991), herein incorporated by reference in its entirety), daily intake of Immuno-LP20 in healthy subjects significantly decreased upper respiratory tract infection (URTI) (FIG. 9C, right panel) in parallel with increased immune functions, as measured by T cell growth (FIG. 8C, left panel).

Another clinical trial (Eur J Nutr. 2020; 59(6): 2641 2649, herein incorporated by reference in its entirety) performed in overweight subjects demonstrated that daily dietary intake of Immuno-LP20 over 12 weeks increased immune functions as measured by T cell growth (FIG. 9D, left panel), decreased inflammation as measured by alanine transaminase (ALT) (FIG. 9D, middle panel), and was beneficial to lipid metabolism as measured by decreased LDL cholesterol levels (FIG. 9D, right panel) in the Immuno-LP20 group compared to the placebo group.

### Example 9: Effect of Bacillus coagulans on Protein Absorption

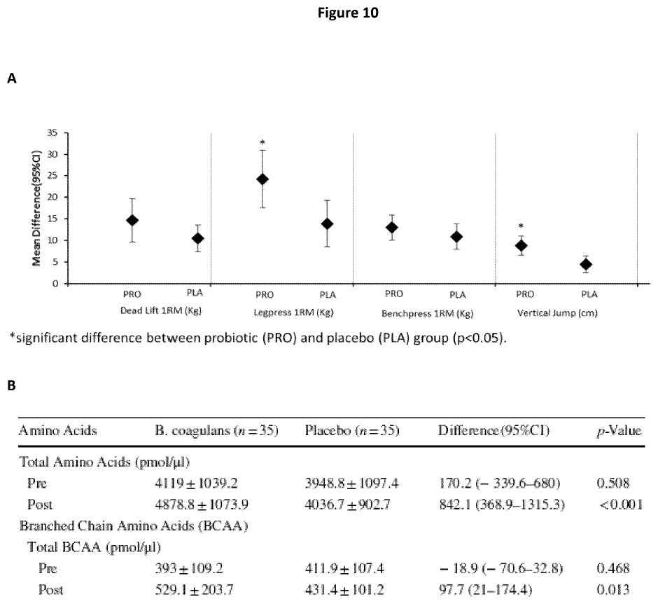

In this double blind, placebo-control trial, resistance-trained males (21.08±2.84 years) were randomized to consume, either 20 g of whey protein powder {80% whey protein concentrate (WPC80), amounting to 15.4 g protein} with 2 billion CFU Bacillus coagulans Unique IS-2 (supplemental group) or 20 g of whey protein powder and lactose instead of Bacillus coagulans (placebo group) once daily for 60 days with a controlled resistance exercise protocol. The whey protein concentrate (WPC-80) given to both groups had a lactose content of 6.8%. Plasma-free amino acids (PFAAs) were determined at baseline, at 30 and 60 days of supplementation. Muscle strength, hypertrophy, VO2 max, and body composition, and other biochemical parameters were assessed at baseline and end line. Probiotic effect was shown on exercise performance as evidenced by an increase in one RM of leg press and vertical jump power by +16.61% (p=0.024) and +7.86% (p=0.007), respectively (FIG. 10A). A positive effect of probiotic Bacillus coagulans Unique IS-2 supplementation was observed on protein absorption as evidenced by an increase in total PFAA by +16.1% (p=0.004) (FIG. 10B, partial results shown). Branched chain amino acids (BCAA) comprising isoleucine (p=0.016), leucine (p=0.001), and valine (p=0.002) were increased by +33.1% in ITT analysis as compared to placebo after 60 days. At 30 days an increase in isoleucine by +35% (p=0.113), leucine by +43% (p=0.032), and valine by +32% (p=0.017) was observed in ITT analysis. In conclusion, significantly increased absorption of BCAA with supplementation of B. coagulans Unique IS-2 along with whey protein and improvement in leg press and vertical jump power was noted indicating the positive effect of the probiotic on muscle power in the lower body. See Tarik et al., The effect of Bacillus coagulans Unique IS-2 supplementation on plasma amino acid levels and muscle strength in resistance trained males consuming whey protein: a double-blind, placebo-controlled study, European Journal of Nutrition, https://doi.org/10.1007/s00394-022-02844-9, herein incorporated by reference in its entirety.

### Example 10: Effect of Heat-Killed Lactobacillus Plantarum L-137 Strain (HK L-137) Supplementation on Periodontal Health

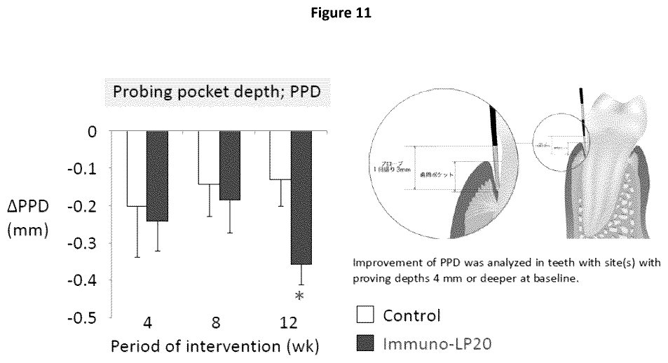

In this randomized, double-blind, placebo-controlled trial, 39 subjects (15 men, 24 women, mean age 66.2 years) with chronic periodontitis undergoing supportive periodontal therapy were enrolled. At baseline, probing pocket deptch (PPD) was assessed. Subsequently, participants were randomly provided with capsules with 50 mg Immuno-LP20 (containing 10 mg of HK L-137) or placebo capsules to be consumed once daily for 12 weeks. After 12 weeks, PPD assessment was repeated. At re-evaluation, PPD was significantly lower in the Immuno-LP20 supplemented group compared to the placebo group (FIG. 11). In conclusion, the consumption of supplements containing heat-killed L. plantarum improves PPD that were greater than 4 mm at baseline. See Iwasaki et al, Daily Intake of Heat-killed Lactobacillus plantarum L-137 Decreases the Probing Depth in Patients Undergoing Supportive Periodontal Therapy, Oral Health Prev Dent. 2016; 14(3):207-14, herein incorporated by reference in its entirety.

### Example 11: Effect of B. coagulans Supplementation on Serum Cholesterol

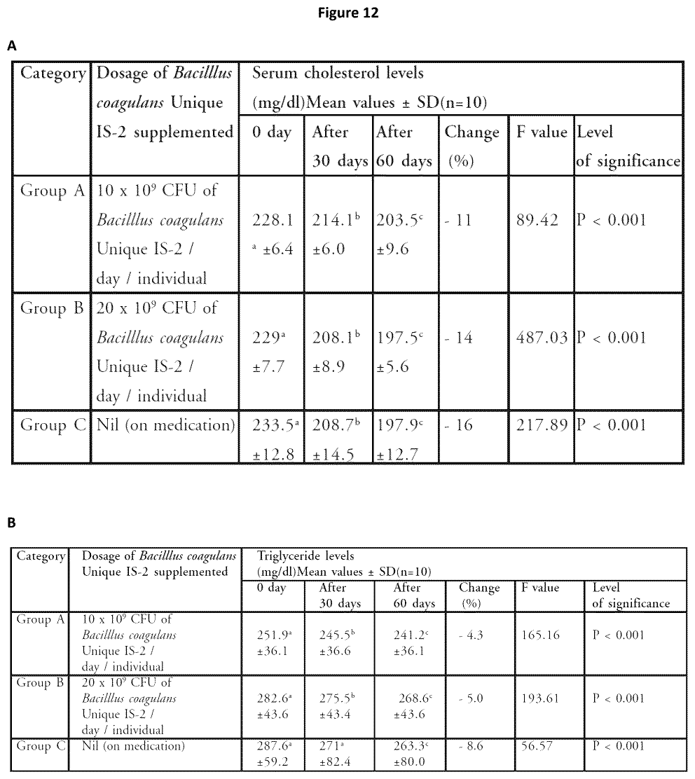

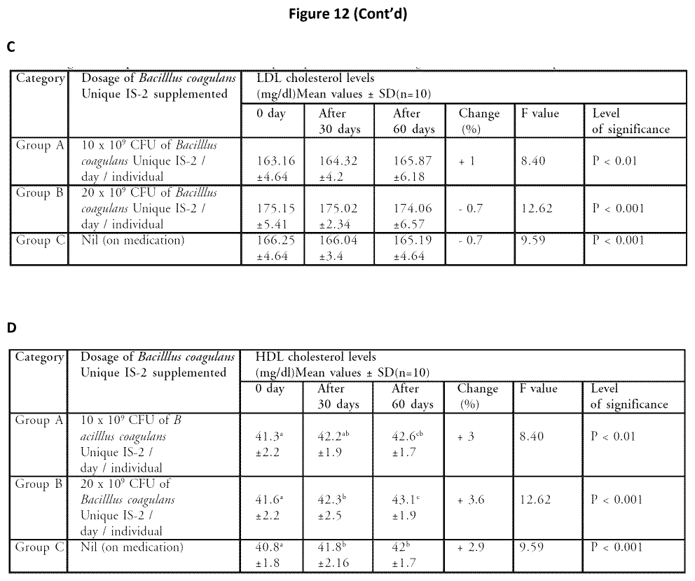

The objective of this study was to evaluate the effect of dietary supplementation of capsules containing Bacillus coagulans Unique IS-2, on serum lipids for 60 days. Thirty hyperlipidemic (having serum cholesterol levels of more than 200 mg/dl) subjects were divided into 3 groups (n=10). Two group subjects were allotted to receive a daily dose of two capsules of probiotic Bacillus coagulans Unique IS2 (10×10⁹ CFU/capsule (Group A) and 20×10⁹ CFU/capsule (Group B)) and third group subjects received standard medication. Serum lipid profiles were taken at 0 days, 30 days, and 60 days of the study period. At the end of study there were slight reductions in total cholesterol (FIG. 12A), triglycerides (FIG. 12B) and LDL (FIG. 12C), and an increase in HDL cholesterol levels (FIG. 12D). These data suggest that the strain Bacillus coagulans IS-2 has the potential of aiding control of the serum cholesterol in humans suffering with hyperlipidemia. Taken from Sudha et al, Effect Of Supplementation Of Probiotic Bacillus coagulans Unique Is-2 On Hypercholesterolemia Subjects: A Clinical Study, International Journal of Probiotics and Prebiotics Vol. 6, No. 2, 2011, herein incorporated by reference in its entirety.

### Example 12: Effect of Lactobacillus plantarum Supplementation on Skin Health

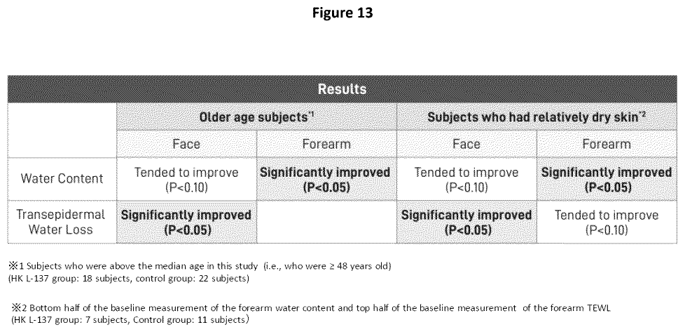

A total of 80 healthy participants (20 men, 60 women; mean age, 47.3 years) were assigned to receive a tablet containing HK L-137 or a placebo tablet daily for 12 weeks. Every 4 weeks, the skin water content and transepidermal water loss (TEWL) were measured at the forearm and face, and participants completed two skin-related questionnaires, the Dermatology Life Quality Index and a self-evaluation. The HK L-137 group tended to show greater increases from baseline of water content at the forearm and larger decreases of TEWL at the face. The total scores of both questionnaires improved significantly more in the HK L-137 group (FIG. 13). Water content and TEWL improved significantly in participants in the HK L-137 group who were above the median age of study participants or had relatively dry skin. These findings suggest that daily HK L-137 intake can improve dry skin, thereby contributing to skin satisfaction. See Yoshitake et al., Beneficial Effect of Heat-Killed Lactiplantibacillus plantarum L-137 on Skin Functions in Healthy Participants: A Randomized, Placebo-Controlled, Double-Blind Study, Front. Med., 6 Jul. 2022, Sec. Dermatology, Volume 9 (2022), herein incorporated by reference in its entirety.

### Example 13: Effect of Bacillus coagulans Supplementation on Dental Caries in Children

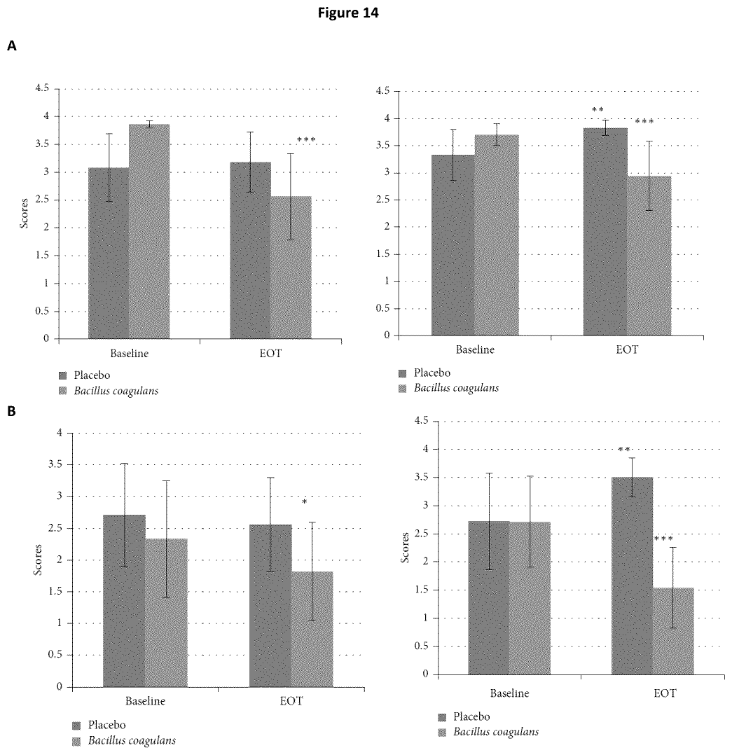

Dental caries or tooth decay is caused by demineralization of the tooth enamel, leading to the breakdown of the enamel causing cavities to be formed. Demineralization of the tooth happens because of the acid secreted by bacteria like mutans streptococci and lactobacilli. Probiotic usage may prevent the overgrowth of these pathogenic microbes, thereby reducing caries activity. In this double-blind, randomized, placebo-controlled study, 48 children with ages ranging from 5 to 15 years were divided into two groups, the probiotic and placebo groups. Chewable tablets with and without probiotic Bacillus coagulans Unique IS-2 were administered for two weeks. Stimulated saliva samples and plaque were collected at baseline and at the end of 14 days to measure the pH, mutans streptococci, and lactobacilli count of saliva and plaque using chairside kits. A statistically significant reduction in mutans streptococci and lactobacilli counts of both saliva and plaque samples was observed in the B. coagulans Unique IS-2 treated group after 14 days of administration compared to the baseline and placebo group (using paired t-test) (FIG. 14). Probiotic Bacillus coagulans Unique IS-2 (2 billion cfu) chewable tablet is effective in reduction and inhibition of caries causing mutans streptococci and lactobacilli levels in saliva and plaque in children. See Sudha et al., Evaluation of the Effect of Probiotic Bacillus coagulans Unique IS-2 on Mutans Streptococci and Lactobacilli Levels in Saliva and Plaque: A Double-Blind, Randomized, Placebo-Controlled Study in Children, International Journal of Dentistry Volume 2020, Article ID 8891708, 8 pages, herein incorporated by reference in its entirety.

### Example 14: Effect of Bacillus coagulans Supplementation on Irritable Bowel Syndrome in Adults and in Children

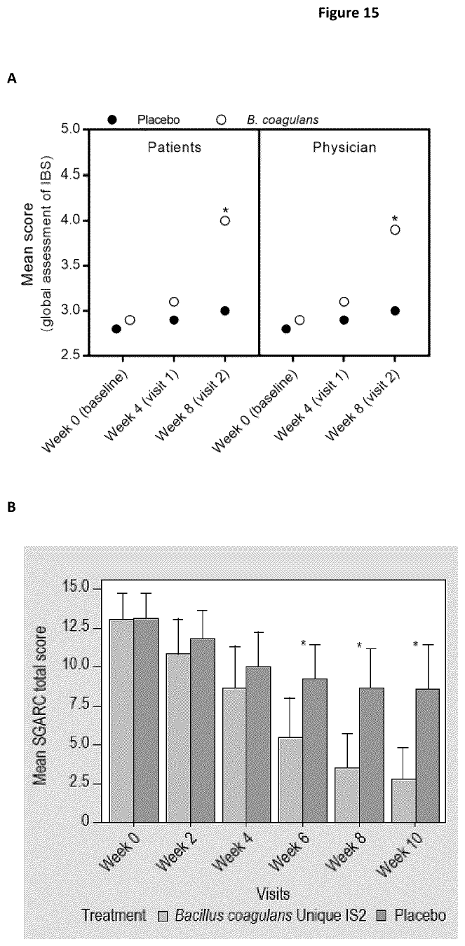

In this study the efficacy of B. coagulans Unique IS-2 in the management of IBS symptoms in adults was investigated. Patients (n=153) fulfilling Rome III criteria were provided placebo capsules for a 2 weeks run-in period. Only patients satisfying compliance criteria (n=136) were randomized (double blind) to receive either B. coagulans Unique IS-2 (2 billion CFU) or placebo capsules daily for 8 weeks. Reduction of abdominal discomfort/pain intensity and increase in complete spontaneous bowel movements were analyzed as primary end points. Other clinical symptoms of IBS and serum cytokines were also evaluated. B. coagulans Unique IS-2 showed significant improvement in primary and secondary endpoints, as compared to placebo. Haematology of both the arms remained normal. No significant changes in pro (IL-6, IL-12, TNF-α, INF-γ) and anti-inflammatory cytokine (IL-10) levels were detected at the end of B. coagulans treatment (8 weeks) as compared to placebo. B. coagulans was well tolerated with no severe adverse events to report. Overall, the results demonstrate that B. coagulans Unique IS-2 is efficacious in the management of IBS symptoms in adults (18-60 years) (FIG. 15A). Taken from Madempudi et al., Randomized clinical trial: the effect of probiotic Bacillus coagulans Unique IS-2 vs. placebo on the symptoms management of irritable bowel syndrome in adults, Scientific Reports (2019) 9:12210, herein incorporated by reference in its entirety.

The efficacy of the probiotic strain, Bacillus coagulans Unique IS-2 in the treatment of Irritable Bowel Syndrome (IBS) was evaluated in children. A total of 141 children of either sex in the age group 4-12 years, diagnosed with IBS according to the Rome III criteria, participated in the double-blind randomised controlled trial. Children received either B. coagulans Unique IS-2 chewable tablets (2×10⁹ CFU, using dextrose as an excipient) or placebo (dextrose excipient only) once daily for eight weeks followed by a two week follow-up period. Reduction in pain intensity as well as other symptoms associated with Irritable Bowel Syndrome like abdominal discomfort, bloating, distension, sense of incomplete evacuation, straining at stool, urgency of bowel movement, passage of gas and mucus, and bowel habit satisfaction were assessed. B. coagulans Unique IS-2 treated group showed a greater reduction in pain scores as evaluated by a weekly pain intensity scale. There was a significant reduction (P<0.0001) in pain intensity in the probiotic treated group (7.6±0.98) as compared to the placebo group (4.2±1.41) by the end of the treatment period (8 weeks). There was also a significant improvement in stool consistency as well as reduction in abdominal discomfort, bloating, staining, urgency, incomplete evacuation and passage of gas. Bowel habit satisfaction and global assessment of relief was also observed in the B. coagulans Unique IS-2 treated group as compared to the placebo group. This study demonstrates the efficacy of B. coagulans Unique IS-2 in reducing the symptoms of Irritable Bowel Syndrome in children in the age group of 4-12 years (FIG. 15B). Talen from Sudha et al., Efficacy of Bacillus coagulans Unique IS-2 in treatment of irritable bowel syndrome in children: a double blind, randomised placebo controlled study, Beneficial Microbes, 2016; in press, herein incorporated by reference in its entirety.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

1. An orally ingestible composition comprising:
an amount of gold kiwifruit powder,
an amount of Bacillus coagulans,
an amount of Bacillus subtilis, and
an amount of heat-treated Lactobacillus plantarum.

2. The composition according to claim 1, wherein the gold kiwifruit powder comprises Livaux®.

3. The composition according to claim 1, wherein the Bacillus coagulans comprises Unique IS-2.

4. The composition according to claim 1, wherein the heat-treated Lactobacillus plantarum comprises L-137.

5. The composition according to claim 1, wherein the Bacillus subtilis comprises BS50.

6. The composition according to claim 1, wherein the ratio of the amount of gold kiwifruit powder:the amount of Bacillus coagulans is between 5:1 and 20:1.

7. The composition according to claim 1, wherein the ratio of the amount of gold kiwifruit powder:the amount of Bacillus subtilis is between 5:1 and 20:1.

8. The composition according to claim 1, wherein the ratio of the amount of gold kiwifruit powder:the amount of Lactobacillus plantarum is between 3:1 and 7:1.

9. The composition according to claim 1, wherein the ratio of the amount of Lactobacillus plantarum:the amount of Bacillus coagulans is between 1.5:1 and 3.5:1.

10. The composition according to claim 1, wherein the ratio of the amount of Lactobacillus plantarum:the amount of Bacillus subtilis is between 1.5:1 and 3.5:1.

11. The composition according to claim 1, wherein the ratio of the amount of Bacillus coagulans:the amount of Bacillus subtilis is between 0.5:1 and 1.5:1.

12. The composition according to claim 1, wherein the orally ingestible composition is in the form of a gummy, a tablet, a capsule, a liquid, a suspension, a powder, or a chew.

13. The composition according to claim 12, wherein the orally ingestible composition is in the form of an individual oral dosage form.

14. The composition according to claim 13, wherein the oral dosage form comprises between about 75 mg and 700 mg gold kiwifruit powder; between about 5 mg and 140 mg heat-treated Lactobacillus plantarum; between about 5 mg and 90 mg Bacillus subtilis; and between about 5 mg and 90 mg Bacillus coagulans.

15. The composition according to claim 13, wherein the oral dosage form comprises between about 500 million CFUs and 9 billion CFUs Bacillus coagulans, and between about 500 million CFUs and 9 billion CFUs Bacillus subtilis.

16. The composition according to claim 13, wherein the oral dosage form comprises about 125 mg gold kiwifruit powder; about 25 mg heat-treated Lactobacillus plantarum; about 10 mg Bacillus subtilis; and about 10 mg Bacillus coagulans.

17. The composition according to claim 13, wherein the oral dosage form comprises about 1 billion CFUs Bacillus coagulans, and about 1 billion CFUs Bacillus subtilis.

18. The composition according to claim 13, wherein the individual oral dosage form is a gummy.

19. A method of increasing the amount of Faecalibacterium prausnitzii within a digestive tract of a human subject, of alleviating IBS symptoms in a human subject suffering from IBS, of supporting protein digestion in a human subject, of reducing bacterial vaginosis symptoms in a human female subject, or of reducing incidence of hepatic encephalopathy in a human subject suffering from liver cirrhosis, wherein the method comprises orally administering, to the human subject, an orally ingestible composition according to claim 1.

20.-24. (canceled)

25. A method of improving cholesterol levels of a human subject, of improving digestive health in a human subject suffering from acute diarrhea, of improving the immune response of a human subject, of improving skin health in a human subject, or of improving protein and amino acid absorption in a human subject, wherein the method comprises orally administering, to the human subject, an orally ingestible composition according to claim 1.

26.-31. (canceled)

32. A method of reducing inflammation caused by plaque-induced gingivitis in a human subject, of improving chronic periodontitis symptoms in a human subject, or of improving oral health and reducing tooth decay in a human subject, wherein the method comprises orally administering, to the human subject, an orally ingestible composition according to claim 1.

33.-43. (canceled)

44. A method of reducing abdominal bloating in a human subject, of reducing burping in a human subject, or of reducing flatulence in a human subject, wherein the method comprises orally administering, to the human subject, an orally ingestible composition according to claim 1.

45.-79. (canceled)
